SEQUENCE LISTING (4 sequences)

SEQ ID NO: 1 - protein, 1572 aa, Homo sapiens
ATGGAGTCAGCTCCGGCAGCCCCCGACCCCGCCGCCAGCGAGCCAGGCAGCAGCGGCGCGGACGCGGCCGCCGGCTCCAGGGAGACCCCGCTGAACCAGGAATCCGCCCGCAAGAGCGAGCCGCCTGCCCCGGTGCGCAGACAGAGCTATTCCAGCACCAGCAGAGGTATCTCAGTAACGAAGAAGACACATACATCTCAAATTGAAATTATTCCATGCAAGATCTGTGGAGACAAATCATCAGGAATCCATTATGGTGTCATTACATGTGAAGGCTGCAAGGGCTTTTTCAGGAGAAGTCAGCAAAGCAATGCCACCTACTCCTGTCCTCGTCAGAAGAACTGTTTGATTGATCGAACCAGTAGAAACCGCTGCCAACACTGTCGATTACAGAAATGCCTTGCCGTAGGGATGTCTCGAGATGCTGTAAAATTTGGCCGAATGTCAAAAAAGCAGAGAGACAGCTTGTATGCAGAAGTACAGAAACACCGGATGCAGCAGCAGCAGCGCGACCACCAGCAGCAGCCTGGAGAGGCTGAGCCGCTGACGCCCACCTACAACATCTCGGCCAACGGGCTGACGGAACTTCACGACGACCTCAGTAACTACATTGACGGGCACACCCCTGAGGGGAGTAAGGCAGACTCCGCCGTCAGCAGCTTCTACCTGGACATACAGCCTTCCCCAGACCAGTCAGGTCTTGATATCAATGGAATCAAACCAGAACCAATATGTGACTACACACCAGCATCAGGCTTCTTTCCCTACTGTTCGTTCACCAACGGCGAGACTTCCCCAACTGTGTCCATGGCAGAATTAGAACACCTTGCACAGAATATATCTAAATCGCATCTGGAAACCTGCCAATACTTGAGAGAAGAGCTCCAGCAGATAACGTGGCAGACCTTTTTACAGGAAGAAATTGAGAACTATCAAAACAAGCAGCGGGAGGTGATGTGGCAATTGTGTGCCATCAAAATTACAGAAGCTATACAGTATGTGGTGGAGTTTGCCAAACGCATTGATGGATTTATGGAACTGTGTCAAAATGATCAAATTGTGCTTCTAAAAGCAGGTTCTCTAGAGGTGGTGTTTATCAGAATGTGCCGTGCCTTTGACTCTCAGAACAACACCGTGTACTTTGATGGGAAGTATGCCAGCCCCGACGTCTTCAAATCCTTAGGTTGTGAAGACTTTATTAGCTTTGTGTTTGAATTTGGAAAGAGTTTATGTTCTATGCACCTGACTGAAGATGAAATTGCATTATTTTCTGCATTTGTACTGATGTCAGCAGATCGCTCATGGCTGCAAGAAAAGGTAAAAATTGAAAAACTGCAACAGAAAATTCAGCTAGCTCTTCAACACGTCCTACAGAAGAATCACCGAGAAGATGGAATACTAACAAAGTTAATATGCAAGGTGTCTACCTTAAGAGCCTTATGTGGACGACATACAGAAAAGCTAATGGCATTTAAAGCAATATACCCAGACATTGTGCGACTTCATTTTCCTCCATTATACAAGGAGTTGTTCACTTCAGAATTTGAGCCAGCAATGCAAATTGATGGGTAA

SEQ ID NO: 2 - protein, 1845 aa, Homo sapiens
ATGACGACCCTGGACTCCAACAACAACACAGGTGGCGTCATCACCTACATTGGCTCCAGTGGCTCCTCCCCAAGCCGCACCAGCCCTGAATCCCTCTATAGTGACAACTCCAATGGCAGCTTCCAGTCCCTGACCCAAGGCTGTCCCACCTACTTCCCACCATCCCCCACTGGCTCCCTCACCCAAGACCCGGCTCGCTCCTTTGGGAGCATTCCACCCAGCCTGAGTGATGACGGCTCCCCTTCTTCCTCATCTTCCTCGTCGTCATCCTCCTCCTCCTTCTATAATGGGAGCCCCCCTGGGAGTCTACAAGTGGCCATGGAGGACAGCAGCCGAGTGTCCCCCAGCAAGAGCACCAGCAACATCACCAAGCTGAATGGCATGGTGTTACTGTGTAAAGTGTGTGGGGACGTTGCCTCGGGCTTCCACTACGGTGTGCACGCCTGCGAGGGCTGCAAGGGCTTTTTCCGTCGGAGCATCCAGCAGAACATCCAGTACAAAAGGTGTCTGAAGAATGAGAATTGCTCCATCGTCCGCATCAATCGCAACCGCTGCCAGCAATGTCGCTTCAAGAAGTGTCTCTCTGTGGGCATGTCTCGAGACGCTGTGCGTTTTGGGCGCATCCCCAAACGAGAGAAGCAGCGGATGCTTGCTGAGATGCAGAGTGCCATGAACCTGGCCAACAACCAGTTGAGCAGCCAGTGCCCGCTGGAGACTTCACCCACCCAGCACCCCACCCCAGGCCCCATGGGCCCCTCGCCACCCCCTGCTCCGGTCCCCTCACCCCTGGTGGGCTTCTCCCAGTTTCCACAACAGCTGACGCCTCCCAGATCCCCAAGCCCTGAGCCCACAGTGGAGGATGTGATATCCCAGGTGGCCCGGGCCCATCGAGAGATCTTCACCTACGCCCATGACAAGCTGGGCAGCTCACCTGGCAACTTCAATGCCAACCATGCATCAGGTAGCCCTCCAGCCACCACCCCACATCGCTGGGAAAATCAGGGCTGCCCACCTGCCCCCAATGACAACAACACCTTGGCTGCCCAGCGTCATAACGAGGCCCTAAATGGTCTGCGCCAGGCTCCCTCCTCCTACCCTCCCACCTGGCCTCCTGGCCCTGCACACCACAGCTGCCACCAGTCCAACAGCAACGGGCACCGTCTATGCCCCACCCACGTGTATGCAGCCCCAGAAGGCAAGGCACCTGCCAACAGTCCCCGGCAGGGCAACTCAAAGAATGTTCTGCTGGCATGTCCTATGAACATGTACCCGCATGGACGCAGTGGGCGAACGGTGCAGGAGATCTGGGAGGATTTCTCCATGAGCTTCACGCCCGCTGTGCGGGAGGTGGTAGAGTTTGCCAAACACATCCCGGGCTTCCGTGACCTTTCTCAGCATGACCAAGTCACCCTGCTTAAGGCTGGCACCTTTGAGGTGCTGATGGTGCGCTTTGCTTCGTTGTTCAACGTGAAGGACCAGACAGTGATGTTCCTAAGCCGCACCACCTACAGCCTGCAGGAGCTTGGTGCCATGGGCATGGGAGACCTGCTCAGTGCCATGTTCGACTTCAGCGAGAAGCTCAACTCCCTGGCGCTTACCGAGGAGGAGCTGGGCCTCTTCACCGCGGTGGTGCTTGTCTCTGCAGACCGCTCGGGCATGGAGAATTCCGCTTCGGTGGAGCAGCTCCAGGAGACGCTGCTGCGGGCTCTTCGGGCTCTGGTGCTGAAGAACCGGCCCTTGGAGACTTCCCGCTTCACCAAGCTGCTGCTCAAGCTGCCGGACCTGCGGACCCTGAACAACATGCATTCCGAGAAGCTGCTGTCCTTCCGGGTGGACGCCCAGTGA

SEQ ID NO: 3 - protein, 1344 aa, Homo sapiens
ATGTCCTTGTGGCTGGGGGCCCCTGTGCCTGACATTCCTCCTGACTCTGCGGTGGAGCTGTGGAAGCCAGGCGCACAGGATGCAAGCAGCCAGGCCCAGGGAGGCAGCAGCTGCATCCTCAGAGAGGAAGCCAGGATGCCCCACTCTGCTGGGGGTACTGCAGGGGTGGGGCTGGAGGCTGCAGAGCCCACAGCCCTGCTCACCAGGGCAGAGCCCCCTTCAGAACCCACAGAGATCCGTCCACAAAAGCGGAAAAAGGGGCCAGCCCCCAAAATGCTGGGGAACGAGCTATGCAGCGTGTGTGGGGACAAGGCCTCGGGCTTCCACTACAATGTTCTGAGCTGCGAGGGCTGCAAGGGATTCTTCCGCCGCAGCGTCATCAAGGGAGCGCACTACATCTGCCACAGTGGCGGCCACTGCCCCATGGACACCTACATGCGTCGCAAGTGCCAGGAGTGTCGGCTTCGCAAATGCCGTCAGGCTGGCATGCGGGAGGAGTGTGTCCTGTCAGAAGAACAGATCCGCCTGAAGAAACTGAAGCGGCAAGAGGAGGAACAGGCTCATGCCACATCCTTGCCCCCCAGGGCTTCCTCACCCCCCCAAATCCTGCCCCAGCTCAGCCCGGAACAACTGGGCATGATCGAGAAGCTCGTCGCTGCCCAGCAACAGTGTAACCGGCGCTCCTTTTCTGACCGGCTTCGAGTCACGCCTTGGCCCATGGCACCAGATCCCCATAGCCGGGAGGCCCGTCAGCAGCGCTTTGCCCACTTCACTGAGCTGGCCATCGTCTCTGTGCAGGAGATAGTTGACTTTGCTAAACAGCTACCCGGCTTCCTGCAGCTCAGCCGGGAGGACCAGATTGCCCTGCTGAAGACCTCTGCGATCGAGGTGATGCTTCTGGAGACATCTCGGAGGTACAACCCTGGGAGTGAGAGTATCACCTTCCTCAAGGATTTCAGTTATAACCGGGAAGACTTTGCCAAAGCAGGGCTGCAAGTGGAATTCATCAACCCCATCTTCGAGTTCTCCAGGGCCATGAATGAGCTGCAACTCAATGATGCCGAGTTTGCCTTGCTCATTGCTATCAGCATCTTCTCTGCAGACCGGCCCAACGTGCAGGACCAGCTCCAGGTAGAGAGGCTGCAGCACACATATGTGGAAGCCCTGCATGCCTACGTCTCCATCCACCATCCCCATGACCGACTGATGTTCCCACGGATGCTAATGAAACTGGTGAGCCTCCGGACCCTGAGCAGCGTCCACTCAGAGCAAGTGTTTGCACTGCGTCTGCAGGACAAAAAGCTCCCACCGCTGCTCTCTGAGATCTGGGATGTGCACGAATGA

SEQ ID NO: 4 - DNA, 3952 bp, synthetic construct
  source 1..3952: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctaaggcaattgagatctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatctcacgtggtcgacgctagcctcgagccaccatggagtcagctccggcagcccccgaccccgccgccagcgagccaggcagcagcggcgcggacgcggccgccggctccagggagaccccgctgaaccaggaatccgcccgcaagagcgagccgcctgccccggtgcgcagacagagctattccagcaccagcagaggtatctcagtaacgaagaagacacatacatctcaaattgaaattattccatgcaagatctgtggagacaaatcatcaggaatccattatggtgtcattacatgtgaaggctgcaagggctttttcaggagaagtcagcaaagcaatgccacctactcctgtcctcgtcagaagaactgtttgattgatcgaaccagtagaaaccgctgccaacactgtcgattacagaaatgccttgccgtagggatgtctcgagatgctgtaaaatttggccgaatgtcaaaaaagcagagagacagcttgtatgcagaagtacagaaacaccggatgcagcagcagcagcgcgaccaccagcagcagcctggagaggctgagccgctgacgcccacctacaacatctcggccaacgggctgacggaacttcacgacgacctcagtaactacattgacgggcacacccctgaggggagtaaggcagactccgccgtcagcagcttctacctggacatacagccttccccagaccagtcaggtcttgatatcaatggaatcaaaccagaaccaatatgtgactacacaccagcatcaggcttctttccctactgttcgttcaccaacggcgagacttccccaactgtgtccatggcagaattagaacaccttgcacagaatatatctaaatcgcatctggaaacctgccaatacttgagagaagagctccagcagataacgtggcagacctttttacaggaagaaattgagaactatcaaaacaagcagcgggaggtgatgtggcaattgtgtgccatcaaaattacagaagctatacagtatgtggtggagtttgccaaacgcattgatggatttatggaactgtgtcaaaatgatcaaattgtgcttctaaaagcaggttctctagaggtggtgtttatcagaatgtgccgtgcctttgactctcagaacaacaccgtgtactttgatgggaagtatgccagccccgacgtcttcaaatccttaggttgtgaagactttattagctttgtgtttgaatttggaaagagtttatgttctatgcacctgactgaagatgaaattgcattattttctgcatttgtactgatgtcagcagatcgctcatggctgcaagaaaaggtaaaaattgaaaaactgcaacagaaaattcagctagctcttcaacacgtcctacagaagaatcaccgagaagatggaatactaacaaagttaatatgcaaggtgtctacattaagagccttatgtggacgacatacagaaaagctaatggcatttaaagcaatatacccagacattgtgcgacttcattttcctccattatacaaggagttgttcacttcagaatttgagccagcaatgcaaattgatgggtaaggtaccggatccgaattcgatatcgcggccgcctcgagcctagggtttaaacctgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgcatgctggggatgcggtgggctctatggccgcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

(19) United States
(12) Patent Application Publication
(10) Pub. No.: US 2024/0148830 A1
(43) Pub. Date: May 9, 2024

(54) Treatment of Neurological Disorder Using NHR

(71) Applicant: Ocugen, Inc., Malvern, PA (US)

(72) Inventor: Arun Kumar Upadhyay, Malvern, PA (US)

(73) Assignee: Ocugen, Inc., Malvern, PA (US)

(21) Appl. No.: 18/503,177

(22) Filed: Nov. 6, 2023

Related U.S. Application Data
(60) Provisional application No. 63/423,481, filed on Nov. 7, 2022.

(51) Int. Cl. A61K 38/17; A61K 48/00; A61P 25/16; A61P 25/28; C12N 15/86

(52) U.S. Cl. CPC A61K 38/1783; A61K 48/0041; A61K 48/0066; A61P 25/16; A61P 25/28; C12N 15/86; C12N 2750/14143

(57) ABSTRACT

The present disclosure provides methods for ameliorating or treating a neurological condition or disease in a subject in need of treatment thereof by administering to the subject a therapeutically effective amount of a composition comprising (i) a recombinant DNA (rDNA), wherein the rDNA comprises a human nuclear hormone receptor (hNHR) gene or a fragment thereof; and (ii) a delivery vehicle adapted for delivering said rDNA to neurological cells or tissues to ameliorate or treat said neurological condition or disease.

### CROSS-REFERENCE TO RELATED APPLICATIONS

This application claims the priority benefit of U.S. Provisional Application No. 63/423,481, filed Nov. 7, 2022, which is incorporated herein by reference in its entirety.

### REFERENCE TO AN ELECTRONIC SEQUENCE LISTING

The contents of the following electronic sequence listing is incorporated herein by reference in its entirety:
Name of the XML File: OCU-000520US_SEQ_ID_LIST.xml;
File Size: 13,305 bytes;
Date of Creation: Nov. 6, 2023.

### FIELD OF THE DISCLOSURE

The present disclosure relates to methods for ameliorating or treating a neurological condition or disease in a subject in need of treatment thereof by administering to the subject a therapeutically effective amount of a composition comprising (i) a recombinant DNA (rDNA), wherein the rDNA comprises a human nuclear hormone receptor (hNHR) gene or a fragment thereof; and (ii) a delivery vehicle adapted for delivering said rDNA to neurological cells or tissues to ameliorate or treat said neurological condition or disease.

### BACKGROUND OF THE DISCLOSURE

Genetic heterogeneity is observed for many Mendelian, single-gene disorders. While environmental influences provide minor contributions, variations in phenotypic outcome are generally attributable to allelic heterogeneity or genetic modifier genes, allelic variants distinct from the mutant gene, which can affect disease onset, progression, and outcome by either increasing or reducing disease severity.

One of the modifier gene therapies under development by the present Applicant is directed to curing retinal degeneration caused by mutations in genes such as NR2E3, RHO, CEP290, PDE6B, etc., for example, by influencing the expression of NR2E3. See, U.S. Pat. No. 9,855,314, issued Jan. 2, 2019, and U.S. Pat. No. 11,351,225, issued Jun. 7, 2022, which are incorporated herein by reference in their entirety. Thus, while some modifier gene therapies are known, there are many other clinical diseases or conditions whose modifier gene therapies are currently not available.

Therefore, there is a continuing need for modifier gene therapies for other clinical diseases or conditions.

### SUMMARY OF THE DISCLOSURE

It has been discovered that nuclear hormone receptors (NHRs) play critical roles in modulating cellular homeostasis through the transcriptional regulation of several downstream genes. Some aspects of the disclosure provide using a modifier gene therapy to treat various genetic diseases arising through a multitude of genetic mutations in various genes but leading to the same result (phenotype) of a diseased condition. A modifier gene alters the phenotype irrespective of the underlying genetic mutation causing the disease.

Without being bound by any theory, it is believed that in some aspects of the disclosure, compositions disclosed herein modulates the expression or activity of a gene, thereby altering the phenotype of the underlying genetic mutation causing the clinical condition or disorder. In some embodiments, the composition of the disclosure reduces the expression or activity of a gene that causes a neurological condition or disorder. Yet in other embodiments, the composition of the disclosure increases the expression or activity of a wild type (or “normal”) gene, thereby ameliorating or treating a neurological condition or disorder.

One particular aspect of the disclosure provides a method for ameliorating or treating a neurological condition or disease in a subject in need of treatment thereof, said method comprising administering to the subject a therapeutically effective amount of a composition comprising:
(i) a recombinant DNA (rDNA) comprising a human nuclear hormone receptor (hNHR) gene or a fragment thereof; and
(ii) a delivery vehicle adapted for delivering said rDNA to neurological cells to ameliorate or treat said neurological condition or disease.

In some embodiments, said delivery vehicle comprises a viral delivery vector. In one particular instance, said viral delivery vector comprises a viral delivery vector (e.g., a capsid protein) associated with adeno-associated virus (AAV), adenovirus, and lentivirus. Yet in other embodiments, said delivery vehicle comprises adeno-associate virus (AAV). Still in some embodiments, rDNA comprises adeno-associated virus inverted terminal repeat (AAV ITR). In one particular embodiment, said AAV ITR comprises AAV2 ITR. Still, in other embodiments, the rDNA further comprises (i) a promotor, (ii) an enhancer, (iii) a polyadenylation moiety, or (iv) a combination thereof. In some instances, said polyadenylation moiety comprises simian virus 40 (SV40) polyadenylation (PolyA) region, bovine growth hormone (bGH) PolyA region, or a combination thereof. In further embodiments, the composition further comprises cytomegalovirus (CMB) promoter or enhancer, elongation factor 1a (EF1a), chicken β-actin (CBA) promoter, CAG promotor, or a combination thereof.

Yet in other embodiments, said delivery vector comprises a non-viral delivery vehicle comprising a nanoparticle, a nanosome, a liposome, a biodegradable polymer complex, or a combination thereof.

In some embodiments, the delivery vehicle is adapted to target the brain using adsorptive ligands such as lectin, cardiolipin, heparin, and cell-penetrating peptides or using transporter ligands such as mannose, glutathione, and different amino acids, or using receptor ligands such as transferrin, OX26 mAb, lactoferrin, apolipoprotein E, polysorbate 80, angiopep-2, candoxin, peptide, RVG29-Rabies virus glycoprotein (29aa peptide), RGD—arginine-glycine-aspartic acid peptide, NGR—asparagine-glycine-arginine peptide. In general, nanoparticles can be targeted to the brain by modifying their surfaces with molecules/ligands specifically recognized by receptors or transporters overexpressed in the brain, such as transferrin, lactoferrin, LDL, nAChR, and αvβ3 integrin receptors, or glucose, glutathione, and amino acids transporters.

Still in other embodiments, the rDNA comprises a cell or tissue-specific promoter. Exemplary cell or tissue-specific promoters that can be used in the invention include, but are not limited to, Human Syn1, MeCP2, NSE, BM88 promoters—broad neuronal expression; CaMKII—glutaminergic neuronal expression specific; DLX5/6 enhancer—GABAergic neurons specific; Tyrosine hydroxylase—Catecholamine neurons specific; Dopamine β hydroxylase (DBH), PRSx8 (synthetic DBH)—Adrenergic and noradrenergic neurons specific; PCP2 (Purkinje cell protein 2)—Purkinje neurons specific; FEV, ETS transcription factor (Ple67)—Serotonergic neurons specific; MCH (melanin-concentrating hormone)—dorsal lateral hypothalamus specific; SLC6A4 (Serotonin transporter Ple198), NR2E1 (ple264)—M\ddot{ū}ller glia specific; GfABC1D (truncated GFAP), Aldh1A1—Astrocytes specific; MBP (Myelin basic protein), MAG (Myelin-associated glycoprotein)—Oligodendrocytes specific; ICAM-2 (Intracellular adhesion molecule 2), CLDN5 (Claudin 5), Tie-2 (TEK, receptor tyrosine kinase), vWF (von Willebrand Factor), FLT1 (Endothelial growth factor receptor)—Endothelial cell-specific; and a combination thereof.

Still in other embodiments, said hNHR gene is selected from the group consisting of NR2E3, NR1C3, NR1D1, RORA, NUPR1, NR2C1, and LXRa. In one particular embodiment, said hNHR gene comprises RORA.

In further embodiments, said neurological condition or disease comprises intellectual developmental disorder, epilepsy, cerebellar ataxia, autism spectrum disorder (ASD), or a combination thereof. In one particular instance, said neurological condition or disease comprises autism spectrum disorder. Yet in another instance, said neurological condition or disease comprises intellectual developmental disorder. Still in another instance, said neurological condition or disease comprises epilepsy. In yet another instance, said neurological condition or disease comprises cerebellar ataxia. In yet another instance, said neurological condition or disease comprises Parkinson's disease. Still another instance, said neurological condition or disease comprises Alzheimer's disease.

In another embodiment, the composition further comprises a cell or tissue-specific promoter for targeted expression in neurological cells or tissues. Exemplary cell or tissue-specific promoters that can be used in the disclosure include, but are not limited to:
Human Syn1, MeCP2, NSE, BM88 promoters—broad neuronal expression;
CaMKII—glutaminergic neuronal expression specific;
DLX5/6 enhancer—GABAergic neurons specific;
Tyrosine hydroxylase—Catecholamine neurons specific;
Dopamine β hydroxylase (DBH), PRSx8 (synthetic DBH)—Adrenergic and noradrenergic neurons specific;
PCP2 (Purkinje cell protein 2)—Purkinje neurons specific;
FEV, ETS transcription factor (Ple67)—Serotonergic neurons specific;
MCH (melanin-concentrating hormone)—dorsal lateral hypothalamus specific;
SLC6A4 (Serotonin transporter Ple198), NR2E1 (ple264)—M\ddot{ū}ller glia specific;
GfABC1D (truncated GFAP), Aldh1A1—Astrocytes specific;
MBP (Myelin basic protein), MAG (Myelin-associated glycoprotein)—Oligodendrocytes specific;
ICAM-2 (Intracellular adhesion molecule 2), CLDN5 (Claudin 5), Tie-2 (TEK, receptor tyrosine kinase), vWF (von Willebrand Factor), FLT1 (Endothelial growth factor receptor)—Endothelial cell-specific; or a combination thereof.

Another aspect of the disclosure provides a method for ameliorating or treating a neurological condition or disease in a subject in need of such a treatment. The method includes administering to the subject a therapeutically effective amount of a composition comprising (i) a human nuclear hormone receptor (hNHR) gene or a fragment thereof, or an mRNA thereof, or a dbDNA thereof; and (ii) an hNHR delivery vehicle, wherein said hNHR gene or a fragment thereof, or an mRNA thereof, or a dbDNA thereof is selected from the group consisting of NR2E3, NR1C3, NR1D1, RORA, NUPR1, NR2C1, and LXRa, or a mRNA thereof, or a dbDNA thereof.

In some embodiments, said delivery vehicle comprises a viral delivery vector or viral capsid protein, wherein said viral delivery vector comprises: a viral delivery vector associated with adeno-associated virus, lentiviral, adenoviral, or HSV1 viral vectors. In other embodiments, the delivery vehicle comprises a nanoparticle or a lipid nanoparticle. Exemplary lipid nanoparticles (LNPs) that can be used in the disclosure include all LNPs known to one skilled in the art of gene therapy. Some exemplary LNPs are disclosed in U.S. Patent Application Publication No. 2022/0184201, which is incorporated herein by reference in its entirety.

Yet in other embodiments, said nanoparticle comprises a liposome, a lipid nanoparticle, a polymeric nanoparticle, a dendrimer, cyclodextrin, a silica nanoparticle, a polymeric complex, a magnetic nanoparticle, a gold nanoparticle, a quantum dot, or a carbon nanotube.

Still in other embodiments, said composition comprises hNHR gene or a fragment thereof, or a plasmid thereof, or an mRNA thereof, or a dbDNA (doggybone) thereof.

In further embodiments, said composition further comprises a pharmaceutically acceptable carrier.

In one particular embodiment, the composition is administered to said subject more than once.

Still in some embodiments, the method of administration of a composition of the disclosure includes Intrathecal, Intraventricular, Intrastriatal, Intrathalamic, or a combination thereof as local routes into CNS. Other methods of administration include systemic routes such as intravenous (IV) injection, intramuscular (IM) injection, etc.

Yet still, in other embodiments, the method comprises administering from about 10⁸ to about 10¹⁴ viral particles to the subject.

Still in other embodiments, composition comprises an rAAV vector. In some instances, the composition further comprises rDNA, which include (i) a promotor, (ii) an enhancer, (iii) a polyadenylation moiety, or (iv) a combination thereof. In one particular instance, said polyadenylation moiety comprises simian virus 40 (SV40) polyadenylation (PolyA) region, bovine growth hormone (bGH) PolyA region, or a combination thereof. In another instance, said vector further comprises cytomegalovirus (CMB) promoter or enhancer, elongation factor 1a (EF1a), chicken β-actin (CBA) promoter, CAG promotor, a cell or tissue-specific promoter for targeted expression in neurological cells or tissues, or a combination thereof.

In further embodiments, the vector is delivered directly to the nerve cells or brain tissue of the subject.

### BRIEF DESCRIPTION OF THE DRAWINGS

FIG. 1 shows various development and homeostasis of several physiological systems that are regulated by RORA.

FIG. 2 shows one particular embodiment of an NHR transgene expression cassette of the disclosure.

### DETAILED DESCRIPTION OF THE INVENTION

The present disclosure relates to using modifier genes known as Nuclear Hormone Receptor (NHR) genes, or a fragment thereof, or an mRNA thereof, or a dbDNA thereof to treat various clinical conditions or diseases. In particular, NHR transgene are used herein to ameliorate or treat a clinical condition associated with a neurological disease or disorder. Nuclear receptors are a class of proteins that are responsible for various activities including, but not limited to, sensing steroids, thyroid hormones, cholesterol, and vitamins. It has been shown that these receptors work with other proteins to regulate the expression of a variety of genes, thereby controlling the development, homeostasis, and metabolism of the organism. As such, these modifier genes can be used to correct defects or genetic disorders that manifest as a wide variety of clinical conditions and/or diseases. It is believed that nuclear receptors bind directly to DNA, thereby regulating the expression of adjacent genes. As such, these receptors are classified as transcription factors. One of the key properties of nuclear receptors that differentiates them from other classes of receptors is their direct control of genomic DNA. There is a wide variety of NHRs known to one skilled in the art including, but not limited to, NR2E3, NR1C3, NR1D1, RORA (i.e., RORα), NUPR1, NR2C1, and LXRa.

The wild type nucleic acid sequence of human RORA (i.e., RORα) mRNA, NR1D1 mRNA, and variant 1 LXRa mRNA are shown in SEQ ID NOS: 1-3, respectively.

Human RORA mRNA sequence (CCDS10177):
(SEQ ID NO: 1)
1 11 21 31 41
ATGGAGTCAG CTCCGGCAGC CCCCGACCCC GCCGCCAGCG AGCCAGGCAG
CAGCGGCGCG GACGCGGCCG CCGGCTCCAG GGAGACCCCG CTGAACCAGG
AATCCGCCCG CAAGAGCGAG CCGCCTGCCC CGGTGCGCAG ACAGAGCTAT
TCCAGCACCA GCAGAGGTAT CTCAGTAACG AAGAAGACAC ATACATCTCA
AATTGAAATT ATTCCATGCA AGATCTGTGG AGACAAATCA TCAGGAATCC
ATTATGGTGT CATTACATGT GAAGGCTGCA AGGGCTTTTT CAGGAGAAGT
CAGCAAAGCA ATGCCACCTA CTCCTGTCCT CGTCAGAAGA ACTGTTTGAT
TGATCGAACC AGTAGAAACC GCTGCCAACA CTGTCGATTA CAGAAATGCC
TTGCCGTAGG GATGTCTCGA GATGCTGTAA AATTTGGCCG AATGTCAAAA
AAGCAGAGAG ACAGCTTGTA TGCAGAAGTA CAGAAACACC GGATGCAGCA
GCAGCAGCGC GACCACCAGC AGCAGCCTGG AGAGGCTGAG CCGCTGACGC
CCACCTACAA CATCTCGGCC AACGGGCTGA CGGAACTTCA CGACGACCTC
AGTAACTACA TTGACGGGCA CACCCCTGAG GGGAGTAAGG CAGACTCCGC
CGTCAGCAGC TTCTACCTGG ACATACAGCC TTCCCCAGAC CAGTCAGGTC
TTGATATCAA TGGAATCAAA CCAGAACCAA TATGTGACTA CACACCAGCA
TCAGGCTTCT TTCCCTACTG TTCGTTCACC AACGGCGAGA CTTCCCCAAC
TGTGTCCATG GCAGAATTAG AACACCTTGC ACAGAATATA TCTAAATCGC
ATCTGGAAAC CTGCCAATAC TTGAGAGAAG AGCTCCAGCA GATAACGTGG
CAGACCTTTT TACAGGAAGA AATTGAGAAC TATCAAAACA AGCAGCGGGA
GGTGATGTGG CAATTGTGTG CCATCAAAAT TACAGAAGCT ATACAGTATG
TGGTGGAGTT TGCCAAACGC ATTGATGGAT TTATGGAACT GTGTCAAAAT
GATCAAATTG TGCTTCTAAA AGCAGGTTCT CTAGAGGTGG TGTTTATCAG
AATGTGCCGT GCCTTTGACT CTCAGAACAA CACCGTGTAC TTTGATGGGA
AGTATGCCAG CCCCGACGTC TTCAAATCCT TAGGTTGTGA AGACTTTATT
AGCTTTGTGT TTGAATTTGG AAAGAGTTTA TGTTCTATGC ACCTGACTGA
AGATGAAATT GCATTATTTT CTGCATTTGT ACTGATGTCA GCAGATCGCT
CATGGCTGCA AGAAAAGGTA AAAATTGAAA AACTGCAACA GAAAATTCAG
CTAGCTCTTC AACACGTCCT ACAGAAGAAT CACCGAGAAG ATGGAATACT
AACAAAGTTA ATATGCAAGG TGTCTACCTT AAGAGCCTTA TGTGGACGAC
ATACAGAAAA GCTAATGGCA TTTAAAGCAA TATACCCAGA CATTGTGCGA
CTTCATTTTC CTCCATTATA CAAGGAGTTG TTCACTTCAG AATTTGAGCC
AGCAATGCAA ATTGATGGGTAA
Human Nr1d1 mRNA (Genbank Accession No. HQ692861.1 (GI:325495532)
(CCDS11361.1)):
(SEQ ID NO: 2)
1 11 21 31 41
ATGACGACCC TGGACTCCAA CAACAACACA GGTGGCGTCA TCACCTACAT
TGGCTCCAGT GGCTCCTCCC CAAGCCGCAC CAGCCCTGAA TCCCTCTATA
GTGACAACTC CAATGGCAGC TTCCAGTCCC TGACCCAAGG CTGTCCCACC
TACTTCCCAC CATCCCCCAC TGGCTCCCTC ACCCAAGACC CGGCTCGCTC
CTTTGGGAGC ATTCCACCCA GCCTGAGTGA TGACGGCTCC CCTTCTTCCT
CATCTTCCTC GTCGTCATCC TCCTCCTCCT TCTATAATGG GAGCCCCCCT
GGGAGTCTAC AAGTGGCCAT GGAGGACAGC AGCCGAGTGT CCCCCAGCAA
GAGCACCAGC AACATCACCA AGCTGAATGG CATGGTGTTA CTGTGTAAAG
TGTGTGGGGA CGTTGCCTCG GGCTTCCACT ACGGTGTGCA CGCCTGCGAG
GGCTGCAAGG GCTTTTTCCG TCGGAGCATC CAGCAGAACA TCCAGTACAA
AAGGTGTCTG AAGAATGAGA ATTGCTCCAT CGTCCGCATC AATCGCAACC
GCTGCCAGCA ATGTCGCTTC AAGAAGTGTC TCTCTGTGGG CATGTCTCGA
GACGCTGTGC GTTTTGGGCG CATCCCCAAA CGAGAGAAGC AGCGGATGCT
TGCTGAGATG CAGAGTGCCA TGAACCTGGC CAACAACCAG TTGAGCAGCC
AGTGCCCGCT GGAGACTTCA CCCACCCAGC ACCCCACCCC AGGCCCCATG
GGCCCCTCGC CACCCCCTGC TCCGGTCCCC TCACCCCTGG TGGGCTTCTC
CCAGTTTCCA CAACAGCTGA CGCCTCCCAG ATCCCCAAGC CCTGAGCCCA
CAGTGGAGGA TGTGATATCC CAGGTGGCCC GGGCCCATCG AGAGATCTTC
ACCTACGCCC ATGACAAGCT GGGCAGCTCA CCTGGCAACT TCAATGCCAA
CCATGCATCA GGTAGCCCTC CAGCCACCAC CCCACATCGC TGGGAAAATC
AGGGCTGCCC ACCTGCCCCC AATGACAACA ACACCTTGGC TGCCCAGCGT
CATAACGAGG CCCTAAATGG TCTGCGCCAG GCTCCCTCCT CCTACCCTCC
CACCTGGCCT CCTGGCCCTG CACACCACAG CTGCCACCAG TCCAACAGCA
ACGGGCACCG TCTATGCCCC ACCCACGTGT ATGCAGCCCC AGAAGGCAAG
GCACCTGCCA ACAGTCCCCG GCAGGGCAAC TCAAAGAATG TTCTGCTGGC
ATGTCCTATG AACATGTACC CGCATGGACG CAGTGGGCGA ACGGTGCAGG
AGATCTGGGA GGATTTCTCC ATGAGCTTCA CGCCCGCTGT GCGGGAGGTG
GTAGAGTTTG CCAAACACAT CCCGGGCTTC CGTGACCTTT CTCAGCATGA
CCAAGTCACC CTGCTTAAGG CTGGCACCTT TGAGGTGCTG ATGGTGCGCT
TTGCTTCGTT GTTCAACGTG AAGGACCAGA CAGTGATGTT CCTAAGCCGC
ACCACCTACA GCCTGCAGGA GCTTGGTGCC ATGGGCATGG GAGACCTGCT
CAGTGCCATG TTCGACTTCA GCGAGAAGCT CAACTCCCTG GCGCTTACCG
AGGAGGAGCT GGGCCTCTTC ACCGCGGTGG TGCTTGTCTC TGCAGACCGC
TCGGGCATGG AGAATTCCGC TTCGGTGGAG CAGCTCCAGG AGACGCTGCT
GCGGGCTCTT CGGGCTCTGG TGCTGAAGAA CCGGCCCTTG GAGACTTCCC
GCTTCACCAA GCTGCTGCTC AAGCTGCCGG ACCTGCGGAC CCTGAACAAC
ATGCATTCCG AGAAGCTGCTGTCCTTCCGGGTGGACGCCCAGTGA
Homo sapiens nuclear receptor subfamily 1 group H member 3
(NR1H3), transcript variant 1, (CCDS7929.1) mRNA (i.e.,
Human LXRa mRNA):
(SEQ ID NO: 3)
1 11
ATGTCCTTGT GGCTGGGGGC CCCTGTGCCT GACATTCCTC CTGACTCTGC
GGTGGAGCTG TGGAAGCCAG GCGCACAGGA TGCAAGCAGC CAGGCCCAGG
GAGGCAGCAG CTGCATCCTC AGAGAGGAAG CCAGGATGCC CCACTCTGCT
GGGGGTACTG CAGGGGTGGG GCTGGAGGCT GCAGAGCCCA CAGCCCTGCT
CACCAGGGCA GAGCCCCCTT CAGAACCCAC AGAGATCCGT CCACAAAAGC
GGAAAAAGGG GCCAGCCCCC AAAATGCTGG GGAACGAGCT ATGCAGCGTG
TGTGGGGACA AGGCCTCGGG CTTCCACTAC AATGTTCTGA GCTGCGAGGG
CTGCAAGGGA TTCTTCCGCC GCAGCGTCAT CAAGGGAGCG CACTACATCT
GCCACAGTGG CGGCCACTGC CCCATGGACA CCTACATGCG TCGCAAGTGC
CAGGAGTGTC GGCTTCGCAA ATGCCGTCAG GCTGGCATGC GGGAGGAGTG
TGTCCTGTCA GAAGAACAGA TCCGCCTGAA GAAACTGAAG CGGCAAGAGG
AGGAACAGGC TCATGCCACA TCCTTGCCCC CCAGGGCTTC CTCACCCCCC
CAAATCCTGC CCCAGCTCAG CCCGGAACAA CTGGGCATGA TCGAGAAGCT
CGTCGCTGCC CAGCAACAGT GTAACCGGCG CTCCTTTTCT GACCGGCTTC
GAGTCACGCC TTGGCCCATG GCACCAGATC CCCATAGCCG GGAGGCCCGT
CAGCAGCGCT TTGCCCACTT CACTGAGCTG GCCATCGTCT CTGTGCAGGA
GATAGTTGAC TTTGCTAAAC AGCTACCCGG CTTCCTGCAG CTCAGCCGGG
AGGACCAGAT TGCCCTGCTG AAGACCTCTG CGATCGAGGT GATGCTTCTG
GAGACATCTC GGAGGTACAA CCCTGGGAGT GAGAGTATCA CCTTCCTCAA
GGATTTCAGT TATAACCGGG AAGACTTTGC CAAAGCAGGG CTGCAAGTGG
AATTCATCAA CCCCATCTTC GAGTTCTCCA GGGCCATGAA TGAGCTGCAA
CTCAATGATG CCGAGTTTGC CTTGCTCATT GCTATCAGCA TCTTCTCTGC
AGACCGGCCC AACGTGCAGG ACCAGCTCCA GGTAGAGAGG CTGCAGCACA
CATATGTGGA AGCCCTGCAT GCCTACGTCT CCATCCACCA TCCCCATGAC
CGACTGATGT TCCCACGGAT GCTAATGAAA CTGGTGAGCC TCCGGACCCT
GAGCAGCGTC CACTCAGAGC AAGTGTTTGC ACTGCGTCTG CAGGACAAAA
AGCTCCCACC GCTGCTCTCT GAGATCTGGG ATGTGCACGA ATGA

It should be appreciated that the scope of the present disclosure also includes allelic variants of SEQ ID NOS: 1-3 known to one skilled in the art. The term “allelic variant” means any of two or more alternative forms of a gene occupying the same chromosomal locus. Allelic variation arises naturally through mutation and may result in polymorphism within populations. Gene mutations can be silent (no change in the encoded polypeptide) or may encode polypeptides having altered amino acid sequences. In some embodiments, the allelic variant is a silent mutation variant.

Furthermore, the scope of the present disclosure also includes nucleic acids that encode a biologically active fragment or a variant of Nr1d1, Rora, or LXRa. A biologically active fragment or variant is a “functional equivalent”—a term that is well understood in the art and is further defined in detail herein. The requisite biological activity of the fragment or variant, using any method disclosed herein or known in the art to establish the activity of a nuclear hormone receptor, has the following activity relative to the wild-type native polypeptide about 50%, about 55%, about 60%, about 65%, about 70%, about 75%, about 80%, about 85%, about 90%, about 95%, about 99%, and any range derivable therein, such as, for example, from about 70% to about 80%, and more preferably from about 81% to about 90%; or even more preferably, from about 91% to about 99%. As used herein, the term “wild-type” refer to one that does not result in undesired phenotype or one that is considered to results in a “normal” phenotype of the typical form of a species as it occurs in nature.

When referring to a numerical value, the terms “about” and “approximately” are used interchangeably herein and refer to being within an acceptable error range for the particular value as determined by one of ordinary skill in the art. Such a value determination will depend at least in part on how the value is measured or determined, e.g., the limitations of the measurement system, i.e., the degree of precision required for a particular purpose. For example, the term “about” can mean within 1 or more than 1 standard deviation, per the practice in the art. Alternatively, the term “about” when referring to a numerical value can mean ±20%, typically ±10%, often ±5%, and more often ±1% of the numerical value. In general, however, where particular values are described in the application and claims unless otherwise stated, the term “about” means within an acceptable error range for the particular value, typically within one standard deviation.

A fragment, in the case of these sequences and all others provided herein, is defined as a part of the whole that is less than the whole. Moreover, a fragment ranges in size from a single nucleotide or amino acid within a polynucleotide or polypeptide sequence to one fewer nucleotide or amino acid than the entire polynucleotide or polypeptide sequence. Finally, a fragment is defined as any portion of a complete polynucleotide or polypeptide sequence that is intermediate between the extremes defined above. For example, fragments of any of the nuclear hormone receptor genes disclosed herein is about 10 nucleotides, 20 nucleotides, 30 nucleotides, 40 nucleotides, 50 nucleotides, 60 nucleotides, 70 nucleotides, 80 nucleotides, 90 nucleotides, 100 nucleotides, 150 nucleotides, 200 nucleotides, 250 nucleotides, 300 nucleotides, 350 nucleotides, 400 nucleotides, 450 nucleotides, 500 nucleotides, 550 nucleotides, 600 nucleotides, 650 nucleotides, 700 nucleotides, 750 nucleotides, 800 nucleotides, 850 nucleotides, 900 nucleotides, 950 nucleotides, 1000 nucleotides, 1100 nucleotides, 1200 nucleotides, 1300 nucleotides, 1400 nucleotides or 1500 nucleotides long.

The term “derivative thereof” refers to a nucleotide sequence having at least about 70%, typically at least about 75%, often at least about 80%, more often at least about 85%, still more often at least about 90%, yet more often at least about 95%, and most often at least about 99% sequence identity or identical sequence as those disclosed in SEQ ID NOS: 1-3.

The terms “identical” and percent “identity,” are used interchangeably herein and in the context of two or more nucleic acids refer to two or more sequences or subsequences that are the same or have a specified percentage of nucleotides that are the same, when compared and aligned for maximum correspondence, as measured using a sequence comparison algorithm such as those described below for example, or by visual inspection.

Alternatively, the phrase “substantially identical,” in the context of two nucleic acid sequences, refers to two or more sequences or subsequences that have at least about 75%, typically at least about 80%, often at least about 85%, more often at least about 90%, and most often at least about 95% or higher nucleotide identity, when compared and aligned for maximum correspondence, as measured using a sequence comparison algorithm such as those described below for example, or by visual inspection. In general, the substantial identity exists over a region of the sequences that is at least about 40-60 nucleotides in length, in other instances over a region at least 60-80 nucleotides in length, in still other instances at least 90-100 nucleotides in length, and in yet other instances the sequences are substantially identical over the full length of the sequences being compared, such as the coding region of a nucleotide for example. Some examples of possible modifications include the insertion of one or more nucleotides in the sequence, the addition of one or more nucleotides in any of the ends of the sequence, or the deletion of one or more nucleotides in any end or inside the sequence. The degree of identity between two polynucleotides can be readily determined using computer algorithms and methods that are widely known for the persons skilled in the art.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection [see generally, Current Protocols in Molecular Biology, (Ausubel, F. M. et al., eds.) John Wiley & Sons, Inc., New York (1987-1999, including supplements such as supplement 46 (April 1999)]. The use of these programs to conduct sequence comparisons is typically conducted using the default parameters specific to each program.

Another example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information. This algorithm involves first identifying high-scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra.). This initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. For identifying whether a nucleic acid is within the scope of the disclosure, the default parameters of the BLAST programs are suitable. The BLASTN program (for nucleotide sequences) uses as defaults a word length (W) of 11, an expectation (E) of 10, M=5, N=−4, and a comparison of both strands. The TBLATN program (using protein sequence for nucleotide sequence) uses as defaults a word length (W) of 3, an expectation (E) of 10, and a BLOSUM 62 scoring matrix. (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which indicates the probability by which a match between two nucleotides would occur by chance. For example, a nucleic acid is considered similar to a reference sequence if the smallest sum probability in a comparison of the test nucleic acid to the reference nucleic acid is less than about 0.1, typically less than about 0.01, and often less than about 0.001.

Another indication that two nucleic acid sequences are substantially identical is that the two molecules hybridize with each other under stringent conditions. “Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence. The phrase “hybridizing specifically to” or “specifically hybridizing to”, refers to the binding, duplexing, or hybridizing of a molecule only to a particular nucleotide sequence under stringent conditions when that sequence is present in a complex mixture (e.g., total cellular) DNA or RNA.

The term “stringent conditions” refers to conditions under which a probe or primer will hybridize to its target subsequence, but no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. In other instances, stringent conditions are chosen to be about 20° C. or 25° C. below the melting temperature of the sequence and a probe with exact or nearly exact complementarity to the target. As used herein, the melting temperature is the temperature at which a population of double-stranded nucleic acid molecules becomes half-dissociated into single strands. Methods for calculating the Tₘ of nucleic acids are well known in the art (see, e.g., Berger and Kimmel (1987) Methods in Enzymology, vol. 152: Guide to Molecular Cloning Techniques, San Diego: Academic Press, Inc. and Sambrook et al. (1989) Molecular Cloning: A Laboratory Manual, 2nd ed., vols. 1-3, Cold Spring Harbor Laboratory), both incorporated herein by reference. As indicated by standard references, a simple estimate of the Tₘ value can be calculated by the equation: Tₘ=81.5+0.41(% G+C), when a nucleic acid is in an aqueous solution at 1 M NaCl (see e.g., Anderson and Young, “Quantitative Filter Hybridization,” in Nucleic Acid Hybridization (1985)). Other references include more sophisticated computations which take structural as well as sequence characteristics into account for the calculation of Tₘ. The melting temperature of a hybrid (and thus the conditions for stringent hybridization) is affected by various factors such as the length and nature (DNA, RNA, base composition) of the probe or primer and the nature of the target (DNA, RNA, base composition, present in solution or immobilized, and the like), and the concentration of salts and other components (e.g., the presence or absence of formamide, dextran sulfate, polyethylene glycol). The effects of these factors are well known and are discussed in standard references in the art, see e.g., Sambrook, supra, and Ausubel, supra. Typically, stringent conditions will be those in which the salt concentration is less than about 1.0 M Na ion, typically about 0.01 to 1.0 M Na ion concentration (or other salts) at pH 7.0 to 8.3 and the temperature is at least about 30° C. for short probes or primers (e.g., 10 to 50 nucleotides) and at least about 60° C. for long probes or primers (e.g., greater than 50 nucleotides). Stringent conditions can also be achieved with the addition of destabilizing agents such as formamide.

In some embodiments, the composition comprising a recombinant nucleic acid, or rDNA, is administered via electroporation. Alternatively, the composition is administered via biodegradable Nile red poly(lactide-co-glycolide) (PLGA) nanoparticle-based gene delivery, small molecule-based gene delivery, naked DNA delivery, viral-based gene delivery, e.g., adeno-associated virus delivery, or genome editing systems, e.g., CRISPR.

The nucleic acid sequences coding for the NHR can be obtained using recombinant methods known in the art, such as, for example, by deriving the gene from a vector known to include the same, or by isolating directly from cells and tissues containing the same, using standard techniques. Alternatively, the gene of interest can be produced synthetically. The nucleic acid can be cloned into a number of types of vectors. For example, the nucleic acid can be cloned into a vector including, but not limited to a plasmid, a phagemid, a phage derivative, an animal virus, a PCR-generated linear DNA sequence, and a cosmid. Vectors of particular interest include expression vectors, replication vectors, probe generation vectors, sequencing vectors and vectors optimized for in vitro transcription.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, carbohydrates, peptides, cationic polymers, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/RNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Optionally, the method further comprises the administration of a pharmaceutically acceptable carrier. The phrase “pharmaceutically acceptable” is art-recognized and refers to compositions, polymers, and other materials and/or dosage forms that are, within the scope of sound medical judgment, suitable for use in contact with the tissues of human beings and animals without excessive toxicity, irritation, allergic response, or other problem or complication, commensurate with a reasonable benefit/risk ratio. For example, pharmaceutically acceptable materials, compositions, or vehicles, such as a liquid or solid filler, diluent, excipient, solvent, or encapsulating material, involved in carrying or transporting any supplement or composition, or component thereof, from one organ, or portion of the body, to another organ, or portion of the body. Each carrier must be “acceptable” in the sense of being compatible with the other ingredients of the supplement and not injurious to the patient. Optionally, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Typically, the polynucleotides and/or other biological agents are purified and/or isolated before administration. As used herein, an “isolated” or “purified” nucleic acid molecule or polynucleotide is substantially free of other cellular material, or culture medium when produced by recombinant techniques, or chemical precursors or other chemicals when chemically synthesized. Purified compounds are at least 60% by weight (dry weight) of the compound of interest. Typically, the preparation is at least 75%, often at least 90%, and most often at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally occurring state. Purified also defines a degree of sterility that is safe for administration to a human subject, e.g., lacking infectious or toxic agents.

Similarly, “substantially pure” means a nucleotide has been separated from the components that naturally accompany it. Typically, the nucleotides are substantially pure when they are at least about 60%, about 70%, about 80%, about 90%, about 95%, or even about 99%, by weight, free from the nucleotides or nucleic acids, and naturally occurring organic molecules with they are naturally associated.

The scope of the disclosure also includes conservatively modified variations of SEQ ID NOS: 1-3. “Conservatively modified variations” of a particular polynucleotide sequence refers to those polynucleotides that encode identical or essentially identical amino acid sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent substitutions” or “silent variations,” which are one species of “conservatively modified variations.” Every polynucleotide sequence described herein which encodes a polypeptide also describes every possible silent variation, except where otherwise noted. Thus, silent substitutions are an implied feature of every nucleic acid sequence which encodes an amino acid. One of skill will recognize that each codon in a nucleic acid (except AUG, which is ordinarily the only codon for methionine) can be modified to yield a functionally identical molecule by standard techniques.

By “isolated nucleic acid” is meant a nucleic acid that is free of the genes which flank it in the naturally-occurring genome of the organism from which the nucleic acid is derived. The term covers, for example: (a) a DNA that is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones. For example, the isolated nucleic acid is a purified cDNA or RNA polynucleotide.

Although the phrase “nucleic acid molecule” primarily refers to the physical nucleic acid and the phrase “nucleic acid sequence” refers to the linear list of nucleotides of the nucleic acid molecule, the two phrases can be used interchangeably.

The terms “effective amount” and “therapeutically effective amount” of a formulation or formulation component mean a sufficient amount of the formulation or component, alone or in a combination, to provide the desired effect. For example, “an effective amount” is meant an amount of a compound, alone or in a combination, required to reduce or prevent ocular disease in a mammal. Ultimately, the attending physician or veterinarian decides the appropriate amount and dosage regimen.

The terms “treating” and “treatment” as used herein refers to the administration of an agent or formulation to a clinically symptomatic individual afflicted with an adverse condition, disorder, or disease to effect a reduction in severity and/or frequency of symptoms, eliminate the symptoms and/or their underlying cause, and/or facilitate improvement or remediation of damage.

The terms “preventing” and “prevention” refer to the administration of an agent or composition to a clinically asymptomatic individual who is susceptible or predisposed to a particular adverse condition, disorder, or disease, and thus relates to the prevention of the occurrence of symptoms and/or their underlying cause.

In some embodiments, the fragments of the present disclosure comprise or consist primarily of the specific domains that are required for or contribute to the functional activity of Nr1d1, Nr2e3, Rora, Nupr1, Nr2c1, or LXRa. For example, nuclear hormone receptors have evolutionarily conserved domains shared with all members of the family, including the highly variable A/B domain, N terminal DNA binding domain, a flexible hinge region, and the ligand-binding and dimerization domain in the C terminus.

Variants encompassed by the present invention include nucleic acid or amino acid sequences comprising the following degrees of sequence identity to Nr1d1, Nr2e3, Rora, Nupr1, Nr2c1, or LXRa: about 50%, about 55%, about 60%, about 65%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, from about 70% to about 80%; typically from about 81% to about 90%; and often from about 91% to about 99% identity.

It should be appreciated that any variations in the coding sequences of the present nucleic acids that, as a result of the degeneracy of the genetic code, express a polypeptide of the same sequence, are included within the scope of this invention.

Any of several known recombinant methods are used to produce a DNA molecule encoding the fragment or variant. For the production of a variant, it is routine to introduce mutations into the coding sequence to generate desired amino acid sequence variants of the invention. Site-directed mutagenesis is a well-known technique for which protocols and reagents are commercially available (e.g., Zoller, M J et al., 1982, Nucl Acids Res 10:6487-6500; Adelman, J P et al., 1983, DNA 2:183-93). These mutations include simple deletions or insertions, systematic deletions, insertions or substitutions of clusters of bases, or substitutions of single bases.

In some aspects, the disclosure includes isolated AAVs. As used herein with respect to AAVs, the term “isolated” refers to an AAV that has been isolated from its natural environment (e.g., from a host cell, tissue, or subject) or artificially produced. Isolated AAVs may be produced using recombinant methods. Such AAVs are referred to herein as “recombinant AAVs”. Recombinant AAVs (rAAVs) may have tissue-specific targeting capabilities, such that a transgene of the rAAV is delivered specifically to one or more predetermined tissue(s). The AAV capsid is an important element in determining these tissue-specific targeting capabilities. Thus, a rAAV having a capsid appropriate for the tissue being targeted can be selected. In some embodiments, the rAAV comprises a capsid protein having an amino acid sequence corresponding to any one of AAV1, AAV2, AAV3, AAV4, AAV5, AAV6, AAV7, AAV8, AAV9, AAV.RhlO, AAV11 and variants thereof. The recombinant AAVs typically includes (i.e., harbors or encapsulates) a recombinant nucleic acid of the disclosure. Methods for obtaining recombinant AAVs having a desired capsid protein are well known in the art (See, for example, U.S. Patent Publication Number 2003/0138772, which is incorporated herein by reference in its entirety). AAV capsid proteins that may be used in the rAAVs of the invention a include, for example, those disclosed in G. Gao, et ah, J. Virol, 78(12):6381-6388 (June 2004); G. Gao, et al, Proc Natl Acad Sci USA, 100(10):6081-6086 (May 13, 2003); U.S. Patent Application Publication Nos. 2003/0138772, 2007/0036760, and 2009/0197338, and WO 2010/138263, all of which relating to AAVs capsid proteins and associated nucleotide and amino acid sequences are incorporated herein by reference. Briefly, the methods involve culturing a host cell which contains a nucleic acid sequence encoding an AAV capsid protein or fragment thereof; a functional rep gene; a recombinant AAV vector composed of AAV inverted terminal repeats (ITRs) and a transgene; and sufficient helper functions to permit packaging of the recombinant AAV vector into the AAV capsid proteins.

Suitable AAVs that may be used in the methods provided herein are disclosed in U.S. Patent Application Publication Nos. 2013/0195801; 2012/0137379, all of which are incorporated herein by reference in their entirety.

The components to be cultured in the host cell to package a rAAV vector in an AAV capsid may be provided to the host cell in trans. Alternatively, any one or more of the required components {e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein. In still another alternative, a selected stable host cell may contain selected component(s) under the control of a constitutive promoter and other selected component(s) under the control of one or more inducible promoters.

The recombinant AAV vector, rep sequences, cap sequences, and helper functions required for producing the rAAV of the invention may be delivered to the packaging host cell using any appropriate NHR genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present invention. See, e.g., K. Fisher et al, J. Virol., 70:520-532 (1993) and U.S. Pat. No. 5,478,745.

In some embodiments, recombinant AAVs may be produced using the triple transfection method, for example, see U.S. Pat. No. 6,001,650. Typically, the recombinant AAVs are produced by transfecting a host cell with a recombinant AAV vector (comprising a NHR transgene) to be packaged into AAV particles, an AAV helper function vector, and an accessory function vector. An AAV helper function vector encodes the “AAV helper function” sequences (e.g., rep and cap), which function in trans for productive AAV replication and encapsidation. In some embodiments, the AAV helper function vector supports efficient AAV vector production without generating any detectable wild-type AAV virions (e.g., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, which are incorporated by reference herein in their entirety. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. Such techniques can be used to introduce one or more exogenous nucleic acids, such as a nucleotide integration vector and other nucleic acid molecules, into suitable host cells. A “host cell” refers to any cell that harbors, or is capable of harboring, a substance of interest. Often a host cell is a mammalian cell, a bacterial, or other suitable cells known to one skilled in the art. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

In some aspects, the disclosure provides isolated cells. As used herein with respect to cell, the term “isolated” refers to a cell that has been isolated from its natural environment (e.g., from a tissue or subject). As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants. As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

The term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or inhibitory RNA (e.g., shRNA, miRNA) from a transcribed gene.

The foregoing methods for packaging recombinant vectors in desired AAV capsids to produce the rAAVs of the disclosure are not meant to be limiting and other suitable methods will be apparent to the skilled artisan having read the present disclosure.

The recombinant nucleic acids of the invention may be recombinant AAV vectors. The recombinant AAV vector may be packaged into a capsid protein and administered to a subject and/or delivered to a selected target cell. “Recombinant AAV (rAAV) vectors” are typically composed of, at a minimum, a transgene and its regulatory sequences. In some embodiments, the transgene also include, 5′- and 3′-AAV inverted terminal repeats (ITRs). The transgene may comprise, as disclosed elsewhere herein, one or more regions that encode one or more NHR.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences range from about 100 bp to about 200 bp, typically from about 110 bp to about 175 bp, often from about 120 bp to about 150 bp, and most often from about 130 bp to about 140 bp in length. In some embodiments, substantially the entire sequences encoding the ITRs are used in the molecule, although some degree of minor modification of these sequences is permissible. The ability to modify these ITR sequences is within the skill of the art. An example of such a molecule employed in the present invention is a “cis-acting” plasmid containing the transgene, in which the selected transgene sequence and associated regulatory elements are flanked by the 5′- and 3′-AAV ITR sequences. The AAV ITR sequences may be obtained from any known AAV, including presently identified mammalian AAV types.

Thus, the recombinant nucleic acids may comprise inverted terminal repeats (ITR) of an AAV serotypes selected from the group consisting of: AAV1, AAV2, AAV5, AAV6, AAV6.2, AAV7, AAV8, AAV9, AAV.Rh10, AAV11 and variants thereof. The recombinant nucleic acids may also include a promoter operably linked with the one or more NHR. The promoter may be tissue-specific promoter, a constitutive promoter or inducible promoter.

In addition to the major elements identified above for the recombinant AAV vector, the vector may also include conventional control elements which are operably linked with elements of the transgene in a manner that permits its transcription, translation and/or expression in a cell transfected with the vector or infected with the virus produced by the disclosure. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (e.g., Kozak consensus sequence); sequences that enhance protein stability; and when desired, sequences that enhance secretion of the encoded product. A number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′-regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly, two or more coding regions are operably linked when they are linked in such a way that their transcription from a common promoter results in the expression of two or more proteins having been translated in frame. In some embodiments, operably linked coding sequences yield a fusion protein. In some embodiments, operably linked coding sequences yield a functional RNA (e.g., miRNA).

For nucleic acids encoding proteins, a polyadenylation sequence generally is inserted following the transgene sequences and before the 3′-AAV ITR sequence. A rAAV construct useful in the present invention may also contain an intron, desirably located between the promoter/enhancer sequence and the transgene. One possible intron sequence is derived from SV-40, and is referred to as the SV-40 T intron sequence. Any intron may be from the β-Actin gene. Another vector element that may be used is an internal ribosome entry site (IRES).

The precise nature of the regulatory sequences needed for gene expression in host cells may vary between species, tissues or cell types, but shall in general include, as necessary, 5′-non-transcribed and 5′-non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer), the SV40 promoter, and the dihydrofolate reductase promoter. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system, the ecdysone insect promoter, the tetracycline-repressible system, the tetracycline-inducible system, the RU486-inducible system and the rapamycin-inducible system. Still other types of inducible promoters which may be useful in this context are those which are regulated by a specific physiological state, e.g., temperature, acute phase, a particular differentiation state of the cell, or in replicating cells only.

In another embodiment, the native promoter, or fragment thereof, for the transgene will be used. In a further embodiment, other native expression control elements, such as enhancer elements, polyadenylation sites or Kozak consensus sequences may also be used to mimic the native expression. In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. In some embodiments, the promoter is a chicken β-actin promoter.

Compositions disclosed herein are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected tissue (e.g., neurons and other nerve cells or tissues) and administration subcutaneously, intraopancreatically, intranasally, parenterally, intravenously, intramuscularly, intrathecally, intracerebrally, orally, intraperitoneally, by inhalation or by another route. Routes of administration may be combined, if desired. Delivery of certain compositions of the disclosure to a subject may be, for example, by administration into the bloodstream of the subject. Administration into the bloodstream may be by injection into a vein, an artery, or any other vascular conduit. Moreover, in certain instances, it may be desirable to deliver the composition of the disclosure to brain tissue, meninges, neuronal cells, glial cells, astrocytes, oligodendrocytes, cereobro spinal fluid (CSF), interstitial spaces and the like. In some embodiments, compositions of the disclosure may be delivered directly to the spinal cord or brain (e.g., prefrontal cortex) by injection into the ventricular region, as well as to the striatum (e.g., the caudate nucleus or putamen of the striatum), and neuromuscular junction, or cerebellar lobule, with a needle, catheter or related device, using neurosurgical techniques known in the art, such as by stereotactic injection.

In certain circumstances it will be desirable to deliver the compositions of the disclosure in suitably formulated pharmaceutical compositions disclosed herein either intrathecally, intracerebrally, intravenously, subcutaneously, intraopancreatically, intranasally, parenterally, intravenously, intramuscularly, orally, intraperitoneally, or by inhalation. It can be appreciated by one skilled in the art that desirable administration of compositions of the disclosure can also include ex vivo administration. In some embodiments, ex vivo administration comprises (1) isolation of cells or tissue(s) of interest from a subject, (2) contacting the cells or tissue(s) with the composition of the disclosure in sufficient amounts to transfect the cells or tissue to provide sufficient levels of gene transfer and expression without undue adverse effect, and (3) transferring cells or tissue back into the subject. In some embodiments, cells or tissues may be cultured ex vivo for several days before and/or after transfection.

Cells or tissues can be isolated from a subject by any suitable method. For example, cells or tissues may be isolated by surgery, biopsy (e.g., biopsy of skin tissue, lung tissue, liver tissue, adipose tissue), or collection of biological fluids such as blood. In some embodiments, cells are isolated from bone marrow. In some embodiments, cells are isolated from adipose tissue. In some embodiments, cells are isolated from a lipoaspirate. Appropriate methods for isolating cells from adipose tissue for ex vivo transfection are known in the art.

In some embodiments, the isolated cells comprise stem cells, pluripotent stem cells, neuroprogenitor cells, lipoaspirate derived stem cells, liver cells {e.g., hepatocytes), hematopoietic stem cells, mesenchymal stem cells, stromal cells, hematopoietic cells, blood cells, fibroblasts, endothelial cells, epithelial cells, or other suitable cells. In some embodiments, cells to be transfected are induced pluripotent stem cells prepared from cells isolated from the subject.

When a viral vector is used, e.g., rAAVs, it may be delivered to a subject in compositions according to any appropriate methods known in the art. The rAAV, which may be suspended in a physiologically compatible carrier (e.g., in a composition), may be administered to a subject, e.g., a human, mouse, rat, cat, dog, sheep, rabbit, horse, cow, goat, pig, guinea pig, hamster, chicken, turkey, or a non-human primate (e.g., Marmoset, Macaque). The compositions of the invention may comprise a rAAV alone, or in combination with one or more other viruses (e.g., a second rAAV encoding having one or more different transgenes). In some embodiments, rAAV vectors expressing NHR (e.g., RORA) are injected in the CSF of the subject both caudally using an IT injection and rostrally using cisterna magna injections. Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. Still others will be apparent to the skilled artisan.

Optionally, the compositions of the invention may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The dose of rAAV virions required to achieve a desired effect or “therapeutic effect,” e.g., the units of dose in vector genomes/per kilogram of body weight (vg/kg), will vary based on several factors including, but not limited to: the route of rAAV administration, the level of gene or RNA expression required to achieve a therapeutic effect, the specific disease or disorder being treated, and the stability of the gene or RNA product. One of skill in the art can readily determine a rAAV virion dose range to treat a subject having a particular disease or disorder based on the aforementioned factors, as well as other factors that are well known in the art. An effective amount of the rAAV is generally in the range of from about 10 μL to about 100 mL of solution containing from about 10⁹ to 10¹⁶ genome copies per subject. Other volumes of solution may be used. The volume used will typically depend, among other things, on the size of the subject, the dose of the rAAV, and the route of administration. For example, for intravenous administration a volume in range of 10 μL to 100 μL, 100 μL to 1 mL, 1 mL to 10 mL, or more may be used. In some cases, a dosage between about 10¹⁰ to 10¹² rAAV genome copies per subject is appropriate. In some embodiments the rAAV is administered at a dose of 10¹⁰, 10¹¹, 10¹², 10¹³, 10¹⁴, or 10¹⁵ genome copies per subject. In some embodiments the rAAV is administered at a dose of 10¹⁰, 10¹¹, 10¹², 10¹³, or 10¹⁴ genome copies per kg.

Formulation of pharmaceutically- acceptable excipients and carrier solutions is well-known to those of skill in the art, as is the development of suitable dosing and treatment regimens for using the particular compositions described herein in a variety of treatment regimens. Typically, these formulations may contain at least about 0.1% of the active ingredient or more, although the percentage of the active ingredient(s) may, of course, be varied and may conveniently be between about 1 or 2% and about 70% or 80% or more of the weight or volume of the total formulation. Naturally, the amount of active ingredient in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the host. The person responsible for administration will, in any event, determine the appropriate dose for the individual host.

Sterile injectable solutions are prepared by incorporating the active composition of the disclosure in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present invention into suitable host cells. In particular, the rAAV vector delivered transgenes may be formulated for delivery either encapsulated in a lipid particle, a liposome, a vesicle, a nanosphere, or a nanoparticle or the like. Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed. Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core. Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (e.g., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), transdermal matrices (U.S. Pat. Nos. 5,770,219 and 5,783,208) and feedback-controlled delivery (U.S. Pat. No. 5,697,899).

### Methods of Treatment

The present disclosure is based upon the discovery of a gene therapy approach in which a modifier gene is administered to neurons or nerve cells either directly or indirectly for treating or preventing various neurological conditions or diseases. Exemplary neurological conditions or diseases that can be treated using the methods of the disclosure include, but are not limited to, intellectual developmental disorder, epilepsy, cerebellar ataxia, autism spectrum disorder (ASD), Parkinson's disease, Alzheimer's disease, neurodegeneration of unknown etiology, or a combination thereof.

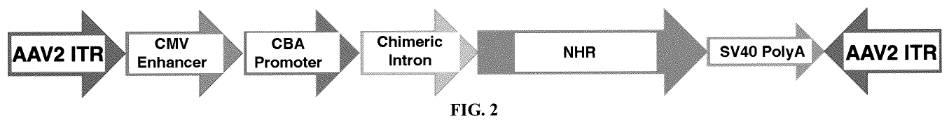

In one embodiment, the disclosure also features an expression vector which includes a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) RNA, and viruses (e.g., lentiviruses, retroviruses, adenoviruses, and adeno-associated viruses) that incorporate the recombinant polynucleotide. The expression vector of the disclosure can also include one or more regulatory elements, e.g., a heterologous promoter. One particular recombinant polynucleotide comprising expression control sequences is illustrated in FIG. 2. In this recombinant DNA or polynucleotide, the transgene hRORA is under transcriptional control of cytomegalovirus (CMV) enhancer and contains the chicken β-actin promoter (CBA) promoter, a Kozak sequence at the transcriptional start site, and the SV40 polyadenylation sequence. As can be seen, the NHR (e.g., hRORA) expression cassette is inserted between two AAV2 ITRs. In particular, the recombinant polynucleotide comprising hRORA illustrated in FIG. 2 comprises nucleotide sequence of SEQ ID NO: 4.

(SEQ ID NO: 4)
cctgcaggca gctgcgcgct cgctcgctca ctgaggccgc
ccgggcgtcg ggcgaccttt ggtcgcccgg cctcagtgag
cgagcgagcg cgcagagagg gagtggccaa ctccatcact
aggggttcct gcggcctaag gcaattgaga tctcgacatt
gattattgac tagttattaa tagtaatcaa ttacggggtc
attagttcat agcccatata tggagttccg cgttacataa
cttacggtaa atggcccgcc tggctgaccg cccaacgacc
cccgcccatt gacgtcaata atgacgtatg ttcccatagt
aacgccaata gggactttcc attgacgtca atgggtggag
tatttacggt aaactgccca cttggcagta catcaagtgt
atcatatgcc aagtacgccc cctattgacg tcaatgacgg
taaatggccc gcctggcatt atgcccagta catgacctta
tgggactttc ctacttggca gtacatctac gtattagtca
tcgctattac catggtcgag gtgagcccca cgttctgctt
cactctcccc atctcccccc cctccccacc cccaattttg
tatttattta ttttttaatt attttgtgca gcgatggggg
cggggggggg gggggggcgc gcgccaggcg gggcggggcg
gggcgagggg cggggcgggg cgaggcggag aggtgcggcg
gcagccaatc agagcggcgc gctccgaaag tttcctttta
tggcgaggcg gcggcggcgg cggccctata aaaagcgaag
cgcgcggcgg gcgggagtcg ctgcgcgctg ccttcgcccc
gtgccccgct ccgccgccgc ctcgcgccgc ccgccccggc
tctgactgac cgcgttactc ccacaggtga gcgggcggga
cggcccttct cctccgggct gtaattagcg cttggtttaa
tgacggcttg tttcttttct gtggctgcgt gaaagccttg
aggggctccg ggagggccct ttgtgcgggg ggagcggctc
ggggggtgcg tgcgtgtgtg tgtgcgtggg gagcgccgcg
tgcggctccg cgctgcccgg cggctgtgag cgctgcgggc
gcggcgcggg gctttgtgcg ctccgcagtg tgcgcgaggg
gagcgcggcc gggggcggtg ccccgcggtg cggggggggc
tgcgagggga acaaaggctg cgtgcggggt gtgtgcgtgg
gggggtgagc agggggtgtg ggcgcgtcgg tcgggctgca
accccccctg cacccccctc cccgagttgc tgagcacggc
ccggcttcgg gtgcggggct ccgtacgggg cgtggcgcgg
ggctcgccgt gccgggcggg gggtggcggc aggtgggggt
gccgggcggg gcggggccgc ctcgggccgg ggagggctcg
ggggaggggc gcggcggccc ccggagcgcc ggcggctgtc
gaggcgcggc gagccgcagc cattgccttt tatggtaatc
gtgcgagagg gcgcagggac ttcctttgtc ccaaatctgt
gcggagccga aatctgggag gcgccgccgc accccctcta
gcgggcgcgg ggcgaagcgg tgcggcgccg gcaggaagga
aatgggcggg gagggccttc gtgcgtcgcc gcgccgccgt
ccccttctcc ctctccagcc tcggggctgt ccgcgggggg
acggctgcct tcggggggga cggggcaggg cggggttcgg
cttctggcgt gtgaccggcg gctctagagc ctctgctaac
catgttcatg ccttcttctt tttcctacag ctcctgggca
acgtgctggt tattgtgctg tctcatcatt ttggcaaaga
atctcacgtg gtcgacgcta gcctcgagcc accatggagt
cagctccggc agcccccgac cccgccgcca gcgagccagg
cagcagcggc gcggacgcgg ccgccggctc cagggagacc
ccgctgaacc aggaatccgc ccgcaagagc gagccgcctg
ccccggtgcg cagacagagc tattccagca ccagcagagg
tatctcagta acgaagaaga cacatacatc tcaaattgaa
attattccat gcaagatctg tggagacaaa tcatcaggaa
tccattatgg tgtcattaca tgtgaaggct gcaagggctt
tttcaggaga agtcagcaaa gcaatgccac ctactcctgt
cctcgtcaga agaactgttt gattgatcga accagtagaa
accgctgcca acactgtcga ttacagaaat gccttgccgt
agggatgtct cgagatgctg taaaatttgg ccgaatgtca
aaaaagcaga gagacagctt gtatgcagaa gtacagaaac
accggatgca gcagcagcag cgcgaccacc agcagcagcc
tggagaggct gagccgctga cgcccaccta caacatctcg
gccaacgggc tgacggaact tcacgacgac ctcagtaact
acattgacgg gcacacccct gaggggagta aggcagactc
cgccgtcagc agcttctacc tggacataca gccttcccca
gaccagtcag gtcttgatat caatggaatc aaaccagaac
caatatgtga ctacacacca gcatcaggct tctttcccta
ctgttcgttc accaacggcg agacttcccc aactgtgtcc
atggcagaat tagaacacct tgcacagaat atatctaaat
cgcatctgga aacctgccaa tacttgagag aagagctcca
gcagataacg tggcagacct ttttacagga agaaattgag
aactatcaaa acaagcagcg ggaggtgatg tggcaattgt
gtgccatcaa aattacagaa gctatacagt atgtggtgga
gtttgccaaa cgcattgatg gatttatgga actgtgtcaa
aatgatcaaa ttgtgcttct aaaagcaggt tctctagagg
tggtgtttat cagaatgtgc cgtgcctttg actctcagaa
caacaccgtg tactttgatg ggaagtatgc cagccccgac
gtcttcaaat ccttaggttg tgaagacttt attagctttg
tgtttgaatt tggaaagagt ttatgttcta tgcacctgac
tgaagatgaa attgcattat tttctgcatt tgtactgatg
tcagcagatc gctcatggct gcaagaaaag gtaaaaattg
aaaaactgca acagaaaatt cagctagctc ttcaacacgt
cctacagaag aatcaccgag aagatggaat actaacaaag
ttaatatgca aggtgtctac attaagagcc ttatgtggac
gacatacaga aaagctaatg gcatttaaag caatataccc
agacattgtg cgacttcatt ttcctccatt atacaaggag
ttgttcactt cagaatttga gccagcaatg caaattgatg
ggtaaggtac cggatccgaa ttcgatatcg cggccgcctc
gagcctaggg tttaaacctg atcataatca gccataccac
atttgtagag gttttacttg ctttaaaaaa cctcccacac
ctccccctga acctgaaaca taaaatgaat gcaattgttg
ttgttaactt gtttattgca gcttataatg gttacaaata
aagcaatagc atcacaaatt tcacaaataa agcatttttt
tcactgcatt ctagttgtgg tttgtccaaa ctcatcaatg
tatcttaacg cgcatgctgg ggatgcggtg ggctctatgg
ccgcgggccg caggaacccc tagtgatgga gttggccact
ccctctctgc gcgctcgctc gctcactgag gccgggcgac
caaaggtcgc ccgacgcccg ggctttgccc gggcggcctc
agtgagcgag cgagcgcgca gctgcctgca gg

A variety of known nucleic acid vectors can be used in these methods, e.g., recombinant viruses, such as a recombinant adeno-associated virus (rAAV), recombinant adenoviruses, recombinant retroviruses, recombinant poxviruses, and other known viruses in the art, as well as plasmids, cosmids, and phages, etc. Many publications well-known in the art discuss the use of a variety of such vectors for the delivery of genes. See, e.g., Ausubel et al., Current Protocols in Molecular Biology, John Wiley & Sons, New York, latest edition; Kay, MA. et al., 2001, Nat. Med., 7:33-40; and Walther W et al., 2000, Drugs 60:249-71).

The compositions of the disclosure also include recombinant DNA, e.g., a recombinant human nuclear hormone receptor (hNHR) gene or a fragment thereof, or an mRNA thereof, or a dbDNA thereof. The regulatory elements may be endogenously found upstream or downstream of the genes, or they may be exogenous regulatory elements that are not found to regulate the genes in nature and are introduced by recombinant DNA techniques known in the art. The regulatory elements can be operably linked to a gene or fragment thereof of the present disclosure, or a gene encoding a protein or fragment thereof of the present disclosure. Methods for assembly of the recombinant vectors are well-known. See, for example, WO 00/15822 and other references cited therein, all of which are incorporated by reference. Upon delivery of the vector to the subject, e.g., to the nerve cells or brain tissue of the subject, the nucleic acid is optionally integrated into the genome of the cells.

The compositions or the rDNAs of the present disclosure can also include appropriate sequences operably linked to the coding sequence or ORF to promote the expression of the nuclear hormone receptors of the present disclosure in a targeted host cell. “Operably linked” sequences include both expression control sequences such as promoters that are contiguous with the coding sequences and expression control sequences that act trans or distally to control the expression of the polypeptide product.

Expression control sequences include appropriate transcription initiation, termination, promoter, and enhancer sequences; efficient RNA processing signals such as splicing and polyadenylation signals; sequences that stabilize cytoplasmic mRNA; sequences that enhance translation efficiency (e.g., Kozak consensus sequence); sequences that enhance nucleic acid or protein stability; and when desired, sequences that enhance protein processing and/or secretion. Many varied expression control sequences, including native and non-native, constitutive, inducible, and/or tissue-specific, are known in the art and may be utilized herein. depending upon the type of expression desired.

Expression control sequences for eukaryotic cells typically include a promoter, an enhancer, such as one derived from an immunoglobulin gene, SV40, CMV, etc., and a polyadenylation sequence which may include splice donor and acceptor sites. The polyadenylation sequence generally is inserted 3′ to the coding sequence and 5′ to the 3′ ITR sequence. PolyA from bovine growth hormone is an example of a suitable sequence.

The promoter may be selected from several constitutive or inducible promoters that can drive the expression of the selected transgene in a nerve cell or brain tissue setting. Typically, a promoter used is “cell-specific”, meaning that it is selected to direct expression of the selected transgene in a particular nerve cell type or brain tissue.

The rAAV used in the present disclosure can be constructed and produced using the materials and methods described herein and those well-known in the art. The methods for producing the construct of this disclosure are conventional and include genetic engineering, recombinant engineering, and synthetic techniques readily understood by the ordinarily skilled artisan.

Briefly, to package an rAAV construct into an rAAV virion, sequences necessary to express AAV rep and AAV cap or functional fragments thereof as well as helper genes essential for AAV production must be present in the host cells. See, for example, U.S. Patent Pub. 2007/0015238, which describes the production of pseudotyped rAAV virion vectors encoding AAV Rep and Cap proteins of different serotypes and AdV transcription products that provide helper functions. For example, AAV rep and cap sequences may be introduced into the host cell in any known manner including, without limitation, transfection, electroporation, liposome delivery, membrane fusion, biolistic delivery of DNA-coated pellets, viral infection, and protoplast fusion.

In another embodiment, the nucleic acids of the disclosure can be delivered via nanoparticles. The nanoparticles are, for example, lipid-based colloidal particles with a diameter of less than 100 nm. Nanoparticles intended for drug and gene delivery can be characterized for various parameters including particle size, size distribution, morphology, zeta potential, drug loading, syringeability and injectability, in vitro drug release, and stability. The formulation of the nanoparticles varies, with lipid composition, the nucleic acid to lipid ratio, and formulation method, depending on the intended use. Nanoparticle assembly methods are known in the art, and as described in Kompella et al., “Nanoparticles for Drug and Gene Delivery in Treating Diseases of the Eye”; Methods in Pharmacology and Toxicology, 2014, pages 291-316, which is incorporated herein by reference in its entirety.

Genome editing systems can also be used to deliver nucleic acids of the present disclosure. Examples of such genome editing systems include, but are not limited to CRISPR/Cas systems, zinc finger nucleases (ZFNS), and transcription activator-like effector nucleases (TALENS). In such systems, the nucleic acids of the disclosure can be readily incorporated into the host cell genome and expressed. In some embodiments, mutated forms of disease-causing genes (i.e., RORα) can be “edited”, or selectively excised, and replaced with any of the nucleic acids described herein. Expression is modulated by endogenous or exogenous regulatory elements, and expressed of these nucleic acids improves or ameliorates the symptoms of the clinical conditions or disease.

The methods and compositions described herein refer to the restoration or normalization of phenotype. As used herein, the “restoration” or “normalization” refers to increasing or decreasing the expression level or activity of defective gene of a subject to a level similar to those that do not suffer from a neurological condition or disease, i.e., a subject that does not display neurological condition or disease disclosed herein. The restoration or normalization of neurons or brain tissue activity can be measured or determined by various tests that are well known to one skilled in the art.

As described in detail below, gene delivery of an NHR gene to neuron cells or brain tissues efficiently ameliorated clinical, morphological, and functional defects associated with various gene defects leading to observed phenotype neurological condition or disease disclosed herein.

Genetic heterogeneity is observed for many Mendelian, single gene disorders. While environmental influences may provide minor contributions, variations in phenotypic outcomes are generally attributable to allelic heterogeneity or genetic modifier genes. Genetic modifiers are allelic variants, distinct from the mutant gene that can alter neurological disease or condition outcome by either increasing or reducing disease severity and affecting disease onset and progression. Identification of genetic modifiers has a significant impact on the prediction of disease progression and the development of new therapeutic strategies.

The data presented herein illustrate the use of modifier genes for treatments for various neurological conditions or diseases. In some embodiments, rescue neurons or brain tissue integrity and function were achieved through a gene therapy approach by delivering a modifier gene rather than replacing the disease-causing gene. The approach described herein identifies genetic modifiers that suppress neurological conditions or diseases caused by several different genes that converge on specific nodes or pathways within a signaling network. As genes function in networks and not singularly, the impact of any gene delivery is on the network as a whole rather than just a single gene. These studies illustrate that viable therapeutic options which have a broad impact emanate from genetic modifier genes that are capable of modulating a disease state by impacting entire gene networks that regulate specific biological processes rather than a single gene.

Administration of the gene modifier ameliorates or treats clinical, morphological, and/or functional defects associated with the primary gene mutation. In one particular embodiment of the disclosure, RAR-related orphan receptor alpha (RORα), also known as NR1F1 (nuclear receptor subfamily 1, group F, member 1) is used in the modifier gene therapy of the disclosure. RORα plays an important role in lipid metabolism, oxidative stress responses and regulates anti-inflammatory responses, etc.

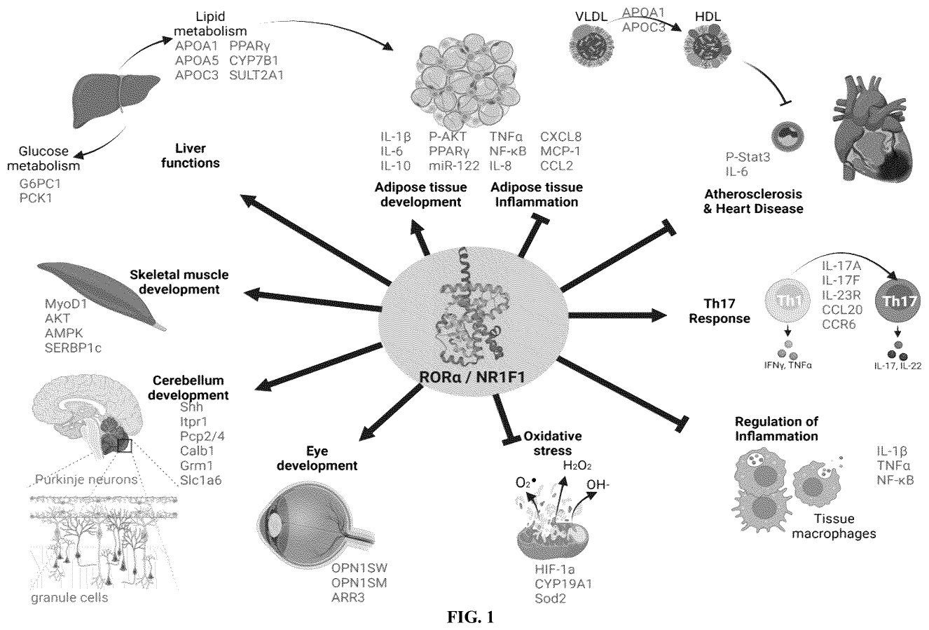

RORα belongs to the NR1 subfamily of nuclear hormone receptors that binds as a monomer or as a homodimer to DNA at the ROR response elements (RORE) containing a core motif 5′-AGGTCA-3′ preceded by a short A-T-rich sequence, upstream of several genes to enhance their expression. RORα is one of the key regulators of embryonic development, cellular differentiation, immunity, circadian rhythm as well as lipid, steroid, xenobiotics, and glucose metabolism. Although RORα has intrinsic transcriptional activity, some natural ligands like oxysterols that act as agonists (25-hydroxycholesterol) or inverse agonists (7-oxygenated sterols), enhance or repress RORα transcriptional activity, respectively. RORα modulates the transcription of several genes by recruiting varying combinations of cofactors to the regulatory regions, depending on the tissue, time, and promoter contexts. Some of the genes regulated by RORα include: (i) the circadian expression of several clock genes, including CLOCK, ARNTL/BMAL1, NPAS2, and CRY1; (ii) cerebellum development like the sonic hedgehog (SHH) gene and other genes involved in calcium-mediated signal transduction; (iii) photoreceptor development like OPN1SW, OPN1SM, and ARR3; (iv) skeletal muscle development with MYOD1; (v) lipid metabolism such as apolipoproteins APOA1, APOA5, APOC3, and PPARγ, and genes CYP7B1 and SULT2A1 encoding phase I and phase II proteins involved in the metabolism of lipids, steroids, and xenobiotics by the liver; (vi) hepatic glucose metabolism through the modulation of G6PC1 and PCK1 by CRY1; (vii) adipocyte differentiation like CEBPB and PPARγ; (viii) the lineage specification of uncommitted CD4 T-helper cells into Th17 cells; (ix) hypoxia signaling like HIF-1α; and (x) anti-inflammatory role like i-κB which inhibits the pro-inflammatory NF-κB signaling. See FIG. 1.

In some embodiments, the composition of the disclosure is administered locally to the neurological cells or neurons, or brain tissues. In another embodiment, the composition is administered intranasally.

The composition is administered at a concentration of 0.001 μg to 100 μg, e.g., 0.01 μg, 0.1 μg, 0.5 μg, 1.0 μg, 1.5 μg, 2.0 μg, 5.0 μg, 10 μg, 20 μg, 30 μg, 40 μg, 50 μg, 60 μg, 70 μg, 80 μg, 90 μg, or 100 μg. The composition is administered in a volume of 0.01 μl to 10 μl, e.g., 0.1 μl, 0.25 μl, 0.5 μl, 1 μl, 2 μl, 2.5 μl, 3 μl, 3.5 μl, 4 μl, 4.5 μl, 5 μl, 6 μl, 7 μl, 8 μl, 9 μl, or 10 μl. The composition is administered once per day, once per week, once per month, every 3 months, every 6 months, or every 12 months. The composition is administered for the duration of 1 day, 1 week, 1 month, 3 months, 6 months, 1 year, 2 years, or 5 years. Alternatively, the composition is administered only once.

The composition comprising a nucleic acid can also be administered via electroporation. Alternatively, the composition can be administered via biodegradable Nile red poly(lactide-co-glycolide) (PLGA) nanoparticle-based gene delivery, small molecule-based gene delivery, naked DNA delivery, viral-based gene delivery, e.g., adeno-associated virus delivery, or genome editing systems, e.g., CRISPR. FIG. 2 shows one particular embodiment of RORA transgene expression cassette that can be used to ameliorate or treat a neurological condition or disease in a subject. In one particular embodiment, this expression cassette is provided in a rAAV vector. In further embodiment, rAAV vector is AAV serotype 5-based (AAV5) vector, AAV2 vector, AAV8 vector, or AAV9 vector. In one particular embodiment, rAAV vector is AAV5 vector. Still in another embodiment, rAAV vector is AAV8 vector.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. GenBank and NCBI submissions indicated by the accession number cited herein are incorporated herein by reference. All other published references, documents, manuscripts, and scientific literature cited herein are incorporated herein by reference. In the case of conflict, the present specification, including definitions, will control. In addition, the materials, methods, and examples are illustrative only and are not intended to be limiting.

### EXAMPLES

The present disclosure relates to using modifier genes known as Nuclear Hormone Receptor (NHR) genes to treat various clinical conditions or diseases. Nuclear receptors are a class of proteins that are responsible for various activities including, but not limited to, sensing steroids, thyroid hormones, cholesterol, and vitamins. It has been shown that these receptors work with other proteins to regulate the expression of a variety of genes, thereby controlling the development, homeostasis, and metabolism of the organism. As such, these modifier genes can be used to correct defects or genetic disorders that manifest as a wide variety of clinical conditions and/or diseases. It is believed that nuclear receptors bind directly to DNA, thereby regulating the expression of adjacent genes. As such, these receptors are classified as transcription factors. One of the key properties of nuclear receptors that differentiates them from other classes of receptors is their direct control of genomic DNA. There is a wide variety of NHRs known to one skilled in the art including, but not limited to RORA (i.e., RORα), NR1D1, and LXRa.

Some aspects of the disclosure provide a method for using a composition that modifies or restore the signaling pathways and/or function of various genes for use in the treatment and/or prevention of various clinical conditions and disorders. For example, the disclosure provides methods of treating clinical diseases or conditions associated with a neurological disorder. Exemplary neurological disorders that can be treated using methods of disclosure include, but are not limited to, intellectual developmental disorder, epilepsy, cerebellar ataxia, autism spectrum disorder (ASD), Parkinson's disease, Alzheimer's disease, neurodegeneration of unknown etiology, or a combination thereof.

Administration of the gene modifier ameliorates clinical, morphological, and functional defects associated with the primary gene mutation. In one particular embodiment of the disclosure, Retinoic acid-related Orphan Receptor Alpha (RORα), also called Nuclear Hormone Receptor 1, Subfamily F1 (NR1F1) is used in the modifier gene therapy of the disclosure. RORα plays an important role in lipid metabolism, oxidative stress responses and regulates anti-inflammatory responses, etc.

RORα belongs to the NR1 subfamily of nuclear hormone receptors that binds as a monomer or as a homodimer to DNA at the ROR response elements (RORE) containing a core motif 5′-AGGTCA-3′ preceded by a short A-T-rich sequence, upstream of several genes to enhance their expression. RORα is one of the key regulators of embryonic development, cellular differentiation, immunity, circadian rhythm as well as lipid, steroid, xenobiotics, and glucose metabolism. Although RORα has intrinsic transcriptional activity, some natural ligands like oxysterols that act as agonists (25-hydroxycholesterol) or inverse agonists (7-oxygenated sterols), enhance or repress RORα transcriptional activity, respectively. RORα modulates the transcription of several genes by recruiting varying combinations of cofactors to the regulatory regions, depending on the tissue, time, and promoter contexts. Some of the genes regulated by RORα include: (i) the circadian expression of several clock genes, including CLOCK, ARNTL/BMAL1, NPAS2, and CRY1; (ii) cerebellum development like the sonic hedgehog (SHH) gene and other genes involved in calcium-mediated signal transduction; (iii) photoreceptor development like OPN1SW, OPN1SM, and ARR3; (iv) skeletal muscle development with MYOD1; (v) lipid metabolism such as apolipoproteins APOA1, APOA5, APOC3, and PPARγ, and genes CYP7B1 and SULT2A1 encoding phase I and phase II proteins involved in the metabolism of lipids, steroids, and xenobiotics by the liver; (vi) hepatic glucose metabolism through the modulation of G6PC1 and PCK1 by CRY1; (vii) adipocyte differentiation like CEBPB and PPARγ; (viii) the lineage specification of uncommitted CD4 T-helper cells into Th17 cells; (ix) hypoxia signaling like HIF-1α; and (x) anti-inflammatory role like i-κB which inhibits the pro-inflammatory NF-κB signaling. See FIG. 1.

RORα staggerer (RORα^{sg}/RORα^{sg}) mouse models: The first homozygous staggerer mouse was observed in 1955 recognized by its staggering gait, mild tremor, hypotonia, and underdeveloped cerebellar cortex with lowered granule and Purkinje cells. Twenty-five years later this mutation was genetically mapped to an interval of 160 kilobases on mouse chromosome 9 containing the RORα gene. This mutation removes an exon encoding part of the RORα ligand-binding domain thereby, generating a truncated protein. Since then, phenotypes involving multiple physiological systems have been observed in the RORα^{sg}/RORα^{sg} mice. Neurological phenotypes include abnormal gait, posture, coordination, and motor learning, with ataxia, hypoactivity, tremors, and associated defects in the cerebellum, cerebrum, and olfactory bulb morphology. The staggerer mice showed abnormal metabolic phenotypes such as elevated prothyrotropin and noradrenaline levels, depletion in the levels of neuronal aspartic acid, taurine, and gamma-aminobutyric acid (GABA), and lowered circulating cholesterol leading to abnormal lipid homeostasis.

Intellectual developmental disorder with or without epilepsy or cerebellar ataxia (IDDECA): The RORα gene in humans is situated on the long (q) arm of chromosome 15 and microdeletions on 15q22.2, overlapping RORα have been reported in individuals with IDDECA. A multi-center study identified three copy-number variant deletions, one disrupting duplication, and nine de novo point mutations (three truncating, one canonical splice site, and five missense mutations) involving the RORα gene in 16 individuals (13 families) with variable neurodevelopmental delay and IDDECA. See, Table 1.

TABLE 1
The genotype and clinical outcome of RORA mutations
in human participants. See, Guissart et al.,
Am J Hum Genet, 2018, 102, pp. 744-759.

| | | Degree of |
|---|---|---|
| | | developmental |
| Mutation | Protein variant | delay or ID |
| c.269G > C | p.Cys90Ser | mild |
| c.275G > C | p.Gly92Ala | mild-moderate |
| c.281A > G | p.Lys94Arg | severe |
| c.425-1G > A | p.Ala142_Leu273del | mild (regression |
| | | at 10 years) |
| c.804_805delGT | p.Ser269Hisfs*13 | mild |
| c.1019delG | p.Arg340Profs*17 | severe |
| c.1225A > C | p.Ser409Arg | mild |
| c.1385G > A | p.Arg462Gln | no ID (IQ 85) |
| c.1498C > T | p.Arg500* | moderate |
| 15q22.2 (59,641,986- | — | mild |
| 61,104,231) x1 | | |
| 15q22.2 (59,641,986- | — | mild |
| 61,104,231) x1 | | |
| 15q22.2 (59,641,986- | — | mild |
| 61,104,231) x1 | | |
| 15q22.2 (59,641,986- | — | mild |
| 61,104,231) x1 | | |
| 15q21.3q22.2 (58,622,268- | — | moderate |
| 62,320,616) x1 | | |
| 15q22.2 (60,809,984- | p.Ser66Trpfs*24 | mild |
| 60,837,029) x1 | | |
| 15q22.2 (60,797,691- | p.Gln315Leufs*51 | mild |
| 60,860,668) x3 | | |

Guissart et al. used the Zebrafish model to recapitulate the neuroanatomical findings found in the 16 affected humans and RORα^{sg} mice. While most mutations show loss of function or haploinsufficiency, 2 missense mutations at the DNA binding domains showed a dominant toxic effect. IDDECA presents either with (A) a cognitive and motor phenotype characterized by a moderate to severe intellectual disability (ID) with ataxia, severe cerebellar vermis hypoplasia, and generalized epilepsy or (B) with a cognitive and behavioral phenotype with autism spectrum disorder (ASD), mild ID, normal cognition and frequently associated with epilepsy. The cerebellar pathology caused by a RORα truncated splice variant (TSV) in the Zebrafish model was rescued using wild-type human RORα mRNA. Id.

The mutations in individuals 6, 7, 8, and 13 (showing ASD) disrupt the RORα ligand-binding domain which agrees with reports of RORα being a candidate for ASD. Analysis has shown that RORα protein is recruited to about 2,544 gene promoter regions across the human genome, specifically to genes controlling neuronal differentiation, adhesion, survival, synaptogenesis, synaptic transmission, plasticity, axon genesis, development of the cortex and cerebellum, cognition, memory, and spatial learning. Independent ChIP-quantitative PCR analyses confirmed the binding of RORα protein to selected ASD-associated gene promoter regions: A2BP1, CYP19A1, ITPR1, NLGN1, and NTRK2. It has been shown that the expression levels of these ASD genes are decreased in RORα-repressed human neuronal cells and prefrontal cortex tissues in individuals with ASD. Additionally, two RORα polymorphisms (rs11639084 and rs4774388) were shown to be associated with ASD risk. Treatment with a synthetic RORα agonist SR1078, reduced repetitive behavior in the BTBR mouse model of autism, indicating that RORα upregulation by a composition disclosed herein is a viable gene therapy method for intellectual developmental disorder with or without epilepsy or cerebellar ataxia (IDDECA) and ASD.

Use of RORα gene therapy as disclosed herein is suitable for treating individuals exhibiting ASD or individuals who are at risk of developing ASD.

Autism spectrum disorder: WT Human RORA rescues cerebellar defect due to RORA-TSV in Zebrafish, whereas R462Q mutant does not. Several studies have linked nuclear receptor defects to autism in humans. RORα polymorphisms (rs11639084 and rs4774388) have been associated with ASD risk. Global methylation profiling revealed that the RORα protein levels were significantly reduced in the brains of individuals with ASD due to epigenetic alterations at the RORa gene. Multiple genes associated with ASD are direct RORα targets and reduction of RORa expression results in reduced expression of these genes leading to ASD. The deficiency of Purkinje cells has consistently been identified as a neuroanatomical abnormality in the brains of individuals with ASD. It has been shown that RORα is critical in the development of Purkinje cells. Thus, increased RORα expression is a treatment for ASD. Individuals with ASD show significant disruptions in their circadian cycles, and RORa plays a role in the regulation of the circadian rhythm. Environmental and metabolic factors of ASD have also been reported to affect RORa expression levels. Sex differences in the expression of RORα and its target genes in the brain have been investigated as a potential contributor to the sex bias in autism. Additionally, the RORαsg mice display behaviors associated with autism including abnormal spatial learning, reduced exploration, limited maze patrolling, and increased perseverative behavior relative to WT mice.

RORα Protein is Reduced In The Autistic Brain: Studying twins where one sibling has autism, and the other did not reveal increased CpG island methylation at the upstream RORα promoter sites of the twin with ASD. Due to this, the lymphoblastoid cell lines (LCL) of the twin with ASD showed reduced expression of RORα protein. FIGS. 4 and 5. Post-mortem examination of age-matched case-control individuals also showed decreased expression of RORα protein in the prefrontal cortex and the cerebellum of autistic individuals. These findings are significant because studies on the RORα^{sg} mice indicate that RORα protein is involved in several processes relevant to autism including Purkinje cell differentiation, cerebellar development, brain lipid homeostasis, protection against oxidative stress and inflammation, and the circadian rhythm.

RORα in Purkinje Cell and Cerebellar Development: Purkinje cells express RORα very early in development which continues during adulthood. In RORα^{sg} mice, most of the Purkinje cells die within the first month of life. The surviving Purkinje cells fail to mature and develop spiny branchlets. RORα is necessary for the retraction of transient dendrites in the early development of Purkinje cells to establish a mature dendritic tree. RORα deficiency in adult mice also creates defects in Purkinje cells such as premature dendritic atrophy and death, higher FoxP2 levels, immature “capuchon” stage of climbing fibers from brain stem olivary neurons, and multi-innervation of Purkinje cells with climbing fibers as opposed to more matured mono-innervation. Therefore, RORα is a terminal differentiation gene that defines the functional properties of a mature Purkinje cell from development to maintenance, throughout its life. The genetic programs in developing Purkinje cells, analyzed daily during mouse prenatal development revealed that RORα bound to the promoter sites and controlled the expression of Shh, Slc1a6, Itpr1, Pcp4, and Pcp2. These RORα target genes provide mitogenic drive and are also required for reciprocal signals between Purkinje, granule, and molecular cells in cerebellar development. Studies in RORα^{sg} mice suggest a possible role of RORα in the expression of cell proliferation, neuronal differentiation, and mature neuron markers (Ki67, DCX, and NeuN, respectively) in the dentate gyms. The dentate gyms is the first region where all sensory modalities converge to form unique representations that bind the different sensory stimuli together, thereby playing a role in learning and memory. Liver X-receptor (LXR)β, a nuclear receptor closely related to RORα has been linked to dentate gyms development abnormalities and autism spectrum disorders. Exogenous RORα expression in RORα^{sg} mice (FIG. 5) partially restored the normal Purkinje cell count and neuronal architecture of the cerebellum indicating ASD can be treated using the composition disclosed herein.

RORα in Brain Lipid Metabolism: Polyunsaturated fatty acids (PUFA), such as omega-6 and omega-3 fatty acids, have been shown to play a role in early brain development. The predominant PUFA species, arachidonic acid (n-6) and docosahexaenoic acid (DHA; n-3) are needed for neuronal growth, synaptogenesis, neuronal survival, and modulation of neurotransmitters. Although abnormal neural lipid metabolism in individuals with ASD has not been extensively investigated, abnormal lipid metabolism has been reported as one of the plasma biomarkers and PUFA interventions in animal models may alleviate autistic-like cognitive and social behaviors. RORα regulates lipoprotein homeostasis and RORα^{sg} mice exhibit aberrant lipid metabolism (reduced serum cholesterol and triglycerides) due to reduced expressions of ApoA1 and ApoC3, respectively. RORα may regulate lipogenesis and mitochondrial fatty acid oxidation by suppressing expressions of peroxisome proliferator-activated receptor-γ (PPARγ), its co-activator PGC1,α and lipin1. Recent reports suggest that RORα deficiency delays all fatty acid accretions during critical periods of brain development, however, deficiency in the omega-3 PUFA species—DHA persists in adult RORα^{sg} mice and is not rescued by dietary DHA supplementation. Similarly, a meta-analysis of case-control cohorts found selectively lower DHA levels in the blood of ASD children (≤12 years) despite no differences in reported dietary intakes with the control group. Although DHA supplementation reversed some impairments in mouse models of ASD (BTBR and serotonin transporter knockout) human trials reporting an increase in blood DHA after dietary supplementation failed to observe improvements in social behavior in children with ASD. This provides additional evidence that RORα deficiency may affect the efficacy of dietary DHA supplementation either by slowing DHA incorporation into, and/or accelerating loss of DHA from brain phospholipids. Therefore, in humans, RORa upregulation by gene therapy is a viable option for individuals with ASD (FIG. 5) in combination with supplements and other medical options for symptom management.

RORα Protects Neurons From Oxidative Stress: Oxidative stress is a common feature in autism cases, which may be further exacerbated due to the presence of genetically susceptible alleles. Limited antioxidant capacity, high energy requirement, and high levels of iron and PUFA in the brain increase its vulnerability to oxidative stress. Postmortem studies on brain tissues of individuals with ASD have shown elevated levels of oxidative damage and reduced antioxidant capacity as compared to age-matched control subjects.

Individuals with ASD show higher levels of lipid hydroperoxide (from fatty acid oxidation); malonyl dialdehyde (from lipid peroxidation); 8-hydroxy-2′-deoxyguanosine (from oxidative DNA damage); protein carbonyl (from protein oxidation); 3-nitrotyrosine (from protein nitration); and carboxyethyl pyrrole (a lipid-derived oxidative protein modification). Overexpression of human RORα1 in cultured mouse cortical neurons increases the expression of the antioxidant proteins glutathione peroxidase 1 (Gpx1) and peroxiredoxin 6 (Prx6), decreases the levels of reactive oxygen species (ROS) (FIG. 5), and protects neurons from apoptosis due to oxidative stressors such as β-amyloid peptide, c₂-ceramide, and H₂O₂. Another study reported that maternal diabetes in mice induced oxidative stress in the brains of their offspring and led to autism-like behavior (ALB). Both the oxidative stress and ALB in the mice offspring were accompanied by downregulation of RORα and its target genes CYP19A1 (aromatase) and Sod2 (superoxide dismutase). Post-natal overexpression of RORα in the offspring rescued both ALB and neuronal oxidative stress, while sh-RNA knockdown had the reverse effect and worsened the ALB.

RORα Protects Neurons From Neuroinflammation: A striking feature common to individuals with ASD is the presence of ongoing neuroinflammation over a broad age range with elevated levels of cytokines and chemokines such as IL-6, TGFβ1, TNFα, CCL2, and CCL17 in the cerebellum and other areas of the brain. Transcriptome organization patterns in the brain of individuals with ASD show abnormalities in gene co-expression networks associated with immune activation. Disrupted monocyte/macrophage function under resting conditions is reported in ASD with decreased production of regulatory (anti-inflammatory) cytokines TGFβ1 and IL-10, and elevated levels of antibodies against cerebellar proteins, all of which are associated with worsening behavioral phenotype. Astrocytes are multifunctional macroglial cells that provide neurons with structural and metabolic support, absorb neurotransmitters, modulate ion concentration, and synaptic transmission, maintain the blood-brain barrier, act as chemosensors, promote myelination, axon regeneration, and drive the molecular oscillations in the circadian clock. Astrocyte role in neuroinflammation has been documented. As effectors of innate immunity in the brain, it is believed that astrocytes are principally activated by the NF-κB signaling pathway and produce high levels of IL-6 by a ROR-dependent mechanism. Although astrocytes from RORα^{sg} mice were reported to have lower resting IL-6 levels than WT mice, upon stimulation by pro-inflammatory cytokines IL-1β and TNFα, the IL-6 levels were significantly higher in RORα^{sg} astrocytes indicative of a pro-neuroinflammatory drive in the absence of RORα (FIG. 5).

RORα Protects Neurons in in-vitro Model of Parkinson's and Alzheimer's Disease Neuroinflammation: To determine whether RORa has a role in neuronal death, Boukhtouche et al (2006) overexpressed the human RORa1 (hRORa1) isoform in neurons either by transient transfection of a plasmid encoding hRORa1 (pSG5-hRORa1) or by infecting cultures with a lentiviral-derived vector encoding hRORa1 (LentihRORa1). hRORa1-overexpressing neurons were then exposed to three different apoptotic stimuli, β-amyloid (Alzheimer's disease model), c2-ceramide (Parkinson's/Alzheimer's disease model) and H2O2 (oxidative stress model), and their survival rate was assessed. Overexpression of hRORa1 not only protects cortical neurons from apoptosis but also significantly improves survival after exposure to apoptotic stimuli. A recent prospective study showed association between sleep duration and Parkinson's disease in carriers of the RORA genotype rs2028122 (Shao et al—2022). Another study (Li et al—2022) showed that RORA was downregulated in Parkinson's disease model and melatonin ameliorated the disease by upregulating RORA expression. Yet another study on nucleoside diphosphate kinase A, a neuroprotective agent in Parkinson's disease revealed its mechanism of action through RORA (Anantha et al—2021). A network analysis study has shown that a notable cross-section of genes differentially expressed in the hippocampi of Alzheimer's disease mice models was found to be linked to RORA (Acquaah-Mensah et al—2015/Darshini et al—2019). Another study identified that RORA regulates microglial cells which mediate Alzheimer's development (Jian et al—2021).

hRORA transgene: A transgene hRORA under transcriptional control of cytomegalovirus (CMV) enhancer and containing the chicken β-actin promoter (CBA) promoter, a Kozak sequence at the transcriptional start site, and the SV40 polyadenylation sequence was produced. The hRORA transgene produced comprised the following sequence of different gene elements (DNA sequence length and name of element): 1-130 (130 bp) left AAV2 ITR; 155-534 (380 bp) CMV enhancer (CAG); 536-813 (278 bp) chicken beta-actin promoter (CAG); 814-1830 (1017 bp) chimeric intron (CAG); 1908-1917 (10 bp) Kozak Sequence; 1914-3485 (1572 bp) hRORA; 3646-3767 (122 bp) SV40 poly A signal sequence; and 3812-3952 (141 bp) right AAV2 ITR.

This transgene hRORA is administered to neurological cells, tissues, and organs to determine therapeutic efficacy of various neurological conditions and diseases.

The foregoing discussion of the invention has been presented for purposes of illustration and description. The foregoing is not intended to limit the invention to the form or forms disclosed herein. Although the description of the invention has included a description of one or more embodiments and certain variations and modifications, other variations and modifications are within the scope of the invention, e.g., as may be within the skill and knowledge of those in the art, after understanding the present disclosure. It is intended to obtain rights that include alternative embodiments to the extent permitted, including alternate, interchangeable, and/or equivalent structures, functions, ranges, or steps to those claimed, whether or not such alternate, interchangeable, and/or equivalent structures, functions, ranges or steps are disclosed herein, and without intending to publicly dedicate any patentable subject matter. All references cited herein are incorporated by reference in their entirety.

What is claimed is:

1. A method for ameliorating or treating a neurological condition or disease in a subject in need of treatment thereof, said method comprising administering to the subject in need of such a treatment a therapeutically effective amount of a therapeutic agent comprising:
(a) a recombinant DNA (rDNA), a recombinant RNA, or a combination thereof of a human nuclear hormone receptor (hNHR) gene or a fragment thereof; and
(b) a delivery vehicle adapted for delivering said hNHR gene or a fragment thereof to a neurological cell, tissue, organ, or a combination thereof to ameliorate or treat said neurological condition or disease.

2. The method of claim 1, wherein said delivery vehicle comprises a viral vector.

3. The method of claim 2, wherein said viral delivery vector comprises a viral vector associated with adeno-associated virus (AAV), adenovirus, and lentivirus.

4. The method of claim 1, wherein said rDNA further comprises (i) a promotor, (ii) an enhancer, (iii) a polyadenylation moiety, or (iv) a combination thereof.

5. The method of claim 4, wherein said polyadenylation moiety comprises simian virus 40 (SV40) polyadenylation (PolyA) region, bovine growth hormone (bGH) PolyA region, or a combination thereof.

6. The method of claim 1, wherein said rDNA further comprises cytomegalovirus (CMV) promoter or enhancer, elongation factor 1a (EF1a), chicken β-actin (CBA) promoter, CAG promotor, or a combination thereof.

7. The method of claim 1, wherein said delivery vector comprises a non-viral delivery vehicle comprising a nanoparticle, a nanosome, a liposome, a biodegradable polymer complex, or a combination thereof.

8. The method of claim 7, wherein said nanoparticle comprises a liposome, a lipid nanoparticle, a polymeric nanoparticle, a dendrimer, cyclodextrin, a silica nanoparticle, a polymeric complex, a magnetic nanoparticle, a gold nanoparticle, a quantum dot, a carbon nanotube, or a combination thereof.

9. The method of claim 1, wherein said hNHR gene is selected from the group consisting of NR1D1, RORA, and LXRa.

10. The method of claim 9, wherein said hNHR gene comprises RORA.

11. The method of claim 1, wherein said neurological condition or disease comprises intellectual developmental disorder, epilepsy, cerebellar ataxia, autism spectrum disorder (ASD), Parkinson's disease, Alzheimer's disease, neurodegeneration of unknown etiology, or a combination thereof.

12. A recombinant adeno-associated virus (AAV) gene therapy particle comprising an AAV capsid protein and a nucleic acid sequence encoding an hRORA protein or a biologically active portion thereof operatively linked to a promoter, and a first and a second AAV inverted terminal repeat (ITR) sequence flanking the sequence encoding the hRORA protein or portion thereof.

13. The recombinant AAV gene therapy particle of claim 12, wherein said nucleic acid sequence further comprises a cytomegalovirus enhancer; a chicken β-actin promoter; a chimeric intron; and a simian virus 40 polyadenylation region.

14. The recombinant AAV gene therapy particle of claim 12, wherein said AAV capsid protein comprises a capsid protein from an AAV serotype selected from the group consisting of: AAV1, AAV2, AAV5, AAV6, AAV6.2, AAV7, AAV8, AAV9, AAVRh10, AAV11 and variants thereof.

15. The recombinant AAV vector of claim 12, wherein said nucleic acid sequence comprises a sequence having at least 95% identity to the nucleic acid sequence of SEQ ID NO:4.

16. A method for ameliorating or treating a neurological condition or disease in a subject in need of treatment thereof, said method comprising administering to the subject in need of such a treatment a therapeutically effective amount of a composition comprising:
a recombinant DNA (rDNA) to ameliorate or treat the neurological condition or disease, wherein said rDNA comprises a nucleic acid sequence having at least 95% identity to the nucleic acid sequence of SEQ ID NO: 4; and
a delivery vehicle adapted for delivering said recombinant DNA to a neurological cell, tissue, organ, or a combination thereof of the subject.

17. The method of claim 16, wherein said delivery vehicle comprises a viral vector.

18. The method of claim 17, wherein said viral vector comprises an adeno-associated virus (AAV) capsid protein.

19. The method of claim 18, wherein said AAV capsid protein comprises a capsid protein from an AAV serotype selected from the group consisting of: AAV1, AAV2, AAV5, AAV6, AAV6.2, AAV7, AAV8, AAV9, AAVRh10, AAV11 and variants thereof.
